(19) United States
(12) Patent Application Publication
(10) Pub. No.: US 2007/0280972 A1
(43) Pub. Date: Dec. 6, 2007

(54) Adhesive solid gel-forming formulations for dermal drug delivery

(75) Inventors: Jie Zhang, Salt Lake City, UT (US); Kevin S. Warner, West Jordan, UT (US); Sanjay Sharma, Sandy, UT (US)

Correspondence Address: THORPE NORTH & WESTERN, LLP., 8180 SOUTH 700 EAST, SUITE 350, SANDY, UT, 84070, US

(73) Assignee: ZARS, Inc.

(21) Appl. No.: 11/796,145

(22) Filed: Apr. 25, 2007

Related U.S. Application Data
(60) Provisional application No. 60/795,091, filed on Apr. 25, 2006.

(51) Int. Cl. A61K 47/06; A61K 31/07; A61K 31/4439; A61K 47/10; A61K 47/14; A61K 47/46; A61P 17/06; A61P 25/30; A61P 31/22; A61P 25/00; A61P 17/00; A61K 47/18; A61K 47/12; A61K 31/56; A61K 31/59

(52) U.S. Cl. 424/400; 514/168; 514/178; 514/343; 514/725; 514/772; 514/783; 514/784; 514/785; 514/788

(57) ABSTRACT

The present invention is drawn to adhesive solid gel-forming formulations, methods of drug delivery, and solidified gel layers for dermal delivery of a drug. The formulation can include a drug, a solvent vehicle, and a gelling agent. The solvent vehicle can include a volatile solvent system having one or more volatile solvent, and a non-volatile solvent system having one or more non-volatile solvent, wherein at least one non-volatile solvent is flux-enabling non-volatile solvent(s) capable of facilitating the delivery of the drug at therapeutically effective rates over a sustained period of time. The formulation can have a viscosity suitable for application to a skin surface prior to evaporation of the volatile solvents system. When applied to the skin, the formulation can form a solidified gel layer after at least a portion of the volatile solvent system is evaporated. The solidified gel layer is can be removed by either peeling or washing using a designated solvent or solvents.

This application claims the benefit of U.S. Provisional Patent Application No. 60/795,091, filed Apr. 25, 2006, which is incorporated herein by reference in its entirety.

### FIELD OF THE INVENTION

The present invention relates generally to systems developed for dermal delivery of drugs. More particularly, the present invention relates to adhesive solid gel-forming formulations having a viscosity suitable for application to a skin surface, and which forms a sustained drug-delivering adhesive-solidified layer on the skin.

### BACKGROUND OF THE INVENTION

Traditional dermal drug delivery systems can generally be classified into two forms: semisolid formulations and dermal patch dosage forms. Semisolid formulations are available in a few different forms, including ointments, creams, foams, pastes, gels, or lotions and are applied topically to the skin. Dermal (including transdermal) patch dosage forms also are available in a few different forms, including matrix patch configurations and liquid reservoir patch configurations. In a matrix patch, the active drug is mixed in an adhesive that is coated on a backing film. The drug-laced adhesive layer is typically directly applied onto the skin and serves both as means for affixing the patch to the skin and as a reservoir or vehicle for facilitating delivery of the drug. Conversely, in a liquid reservoir patch, the drug is typically incorporated into a solvent system which is held by a thin bag, which can be a thin flexible container. The thin bag can include a permeable or semi-permeable membrane surface that is coated with an adhesive for affixing the membrane to the skin. The membrane is often referred to as a rate limiting membrane (although it may not actually be rate limiting in the delivery process in all cases) and can control transport of the drug from within the thin bag to the skin for dermal delivery.

While patches and semisolid formulations are widely used to deliver drugs into and through the skin, they both have significant limitations. For example, most semisolid formulations usually contain solvent(s), such as water and ethanol, which are volatile and thus evaporate shortly after application. The evaporation of such solvents can cause a significant decrease or even termination of dermal drug delivery, which may not be desirable in many cases. Additionally, semisolid formulations are often “rubbed into” the skin, which does not necessarily mean the drug formulation is actually delivered into the skin. Instead, this phrase often means that a very thin layer of the drug formulation is applied onto the surface of the skin. Such thin layers of traditional semisolid formulations applied to the skin may not contain sufficient quantity of active drug to achieve sustained delivery over long periods of time. Additionally, traditional semisolid formulations are often subject to unintentional removal due to contact with objects such as clothing, which may compromise the sustained delivery and/or undesirably soil clothing. Drugs present in a semisolid formulation may also be unintentionally delivered to persons who come in contact with a patient undergoing treatment with a topical semisolid formulation.

With respect to matrix patches, in order to be delivered appropriately, a drug should have sufficient solubility in the adhesive, as primarily only dissolved drug contributes to the driving force required for skin permeation. Unfortunately, when the solubility in an adhesive is too low adequate skin permeation driving force over sustained period of time is not generated. In addition, many ingredients, e.g., liquid solvents and permeation enhancers, which could be used to help dissolve the drug or increase the skin permeability, may not be able to be incorporated into many adhesive matrix systems in sufficient quantities to be effective. For example, at functional levels, most of these materials may adversely alter the wear properties of the adhesive. As such, the selection and allowable quantities of additives, enhancers, excipients, or the like in adhesive-based matrix patches can be limited. To illustrate, for many drugs, optimal transdermal flux can be achieved when the drug is dissolved in certain liquid solvent systems, but a thin layer of adhesive in a typical matrix patch often cannot hold enough appropriate drug and/or additives to be therapeutically effective. Further, the properties of the adhesives, such as coherence and tackiness, can also be significantly changed by the presence of liquid solvents or enhancers.

Regarding liquid reservoir patches, even if a drug is compatible with a particular liquid or semisolid solvent system carried by the thin bag of the patch, the solvent system still has to be compatible to the adhesive layer coated on the permeable or semi-permeable membrane; otherwise the drug may be adversely affected by the adhesive layer or the drug/solvent system may reduce the tackiness of the adhesive layer. In addition to these dosage form considerations, reservoir patches are bulkier and usually are more expensive to manufacture than matrix patches.

Another shortcoming of dermal (including transdermal) patches is that they are usually neither stretchable nor flexible, as the backing film (in matrix patches) and the thin fluid bag (in reservoir patches) are typically made of polyethylene or polyester, both of which are relatively non-stretchable materials. If the patch is applied to a skin area that is significantly stretched during body movements, such as a joint, separation between the patch and skin may occur thereby compromising the delivery of the drug. In addition, a patch present on a skin surface may hinder the expansion of the skin during body movements and cause discomfort. For these additional reasons, patches are not ideal dosage forms for skin areas subject to expansion, flexing and stretching during body movements.

It is known that in order for a drug to be absorbed dermally at sufficient therapeutic rates, it typically needs to be dissolved in an appropriate solvent vehicle. The reservoir solution in a reservoir patch and adhesive in a drug-in-adhesive patch are examples of such solvent vehicles. In reservoir and drug-in-adhesive patches, the reservoir enclosure and the backing film, respectively, protect the solvent vehicle against undesirable removal by objects such as clothing and thus enable sustained dermal delivery of the drug. Therefore, dermal patches can be viewed as nothing more than means to securely maintain the drug-containing solvent vehicle on the skin for a sustained period of time. However, the material cost of the reservoir enclosure and the backing film is one of the reasons why a patch is usually much more expensive than a semisolid product for the delivery of the same drug. Patches usually are also less comfortable to wear and are less flexible in coverage area than the semisolid dosage forms. Traditional semi-solid dosage forms such as gels, ointments, creams may also contain such solvent vehicles. However, as mentioned, solvent vehicles in the traditional semisolid dosage forms are not protected against undesired removal, which is one of the reasons why many semisolid products have to be applied multiple times a day.

In view of the shortcomings of many of the current delivery systems, it would be desirable to provide systems, formulations, and/or methods that can i) provide sustained drug delivery over long periods of time; ii) are not vulnerable to unintentional removal by contact with clothing, other objects, or people for the duration of the application time; iii) can be applied to a skin area subject to stretching and expansion without causing discomfort or poor contact to skin; and/or iv) can be easily removed after application and use.

### SUMMARY OF THE INVENTION

In accordance with embodiments of the present invention, it would be advantageous to provide formulations and convenient methods for securely keeping a drug-containing liquid solvent vehicle on the skin for a sustained period of time, without the shortcomings of patches. More specifically, it would be advantageous to provide dermal delivery formulations, systems, and/or methods in the form of solid gel-forming compositions or formulations having a viscosity suitable for application to the skin surface and which form a drug-delivering solidified layer on the skin that is easily removable, by peeling off or washing off with a solvent, after use. In accordance with this, a solid gel-forming formulation for dermal delivery of a drug can comprise a drug, a solvent vehicle, and a gelling agent. The solvent vehicle can comprise a volatile solvent system having one or more volatile solvent(s) and a non-volatile solvent system having one or more non-volatile solvent(s), wherein the non-volatile solvent system comprises at least one flux-enabling non-volatile solvent (to be defined later) for the drug such that the drug can be delivered in therapeutically effective amounts over a sustained period of time, even after most of the volatile solvent(s) is evaporated. The formulation can have viscosity suitable for application to the skin surface prior to evaporation of at least one volatile solvent, and can further be configured such that when applied to the skin surface, the formulation forms a solidified (solid gel) layer after at least a portion of the volatile solvent(s) is evaporated.

In an alternative embodiment, a method of dermally delivering a drug to, into, or through the skin can comprise applying an adhesive solid gel-forming formulation to a skin surface of the subject, dermally delivering the drug from the solidified layer over a period of time and at desired rates, and removing the solidified layer from the skin after a period of time has elapsed or the desired quantity of the drug has been delivered. The adhesive formulation can include a drug, a solvent vehicle, and a gelling agent. The solvent vehicle can comprise a volatile solvent system having one or more volatile solvent and a non-volatile solvent system having one or more non-volatile solvent(s), wherein at least one of the non-volatile solvent(s) or the mixture of non-volatile solvents is flux-enabling. The formulation can have a viscosity suitable for application to a skin surface prior to evaporation of the volatile solvent. When the formulation is applied to the skin surface, the formulation can form a solidified (solid gel) layer after at least a portion of the volatile solvent system evaporates.

In another embodiment, a method of preparing an adhesive solidified formulation for dermal drug delivery can comprise steps of selecting a drug suitable for dermal delivery; selecting or formulating a non-volatile solvent or a mixture of non-volatile solvents that is flux-enabling for the selected drug, selecting a gelling agent that is compatible with the drug and the non-volatile solvent, selecting or formulating a volatile solvent system that is compatible with the drug, the non-volatile solvent and the gelling agent; and formulating all above ingredients into an adhesive solid gel-forming formulation. The adhesive solid gel-forming formulation can have a viscosity suitable for application to a skin surface prior to evaporation of the volatile solvent system, and can be applied to the skin surface where it forms a solidified layer after at least a portion of the volatile solvent system is evaporated. In this embodiment, the drug continues to be delivered at a therapeutically effective amount after the volatile solvent system is substantially evaporated.

In still another embodiment, a solidified layer for delivering a drug can comprise a drug, a non-volatile solvent system, and a gelling agent. The non-volatile solvent system can include at least one flux-enabling non-volatile solvent or a mixture of non-volatile solvents that are flux-enabling. Further, the solidifed layer can be stretched in at least one direction by 5%, or even 10%, without breaking, cracking, or separation from a skin surface to which the solidified layer is applied.

Additional features and advantages of the invention will be apparent from the following detailed description and figures which illustrate, by way of example, features of the invention.

### BRIEF DESCRIPTION OF THE DRAWINGS

FIG. 1 is a graphical representation of the cumulative amount of diclofenac delivered transdermally across human cadaver skin over time from a solidified gel formulation in accordance with embodiments of the present invention where steady-state delivery is shown over 28 hours.

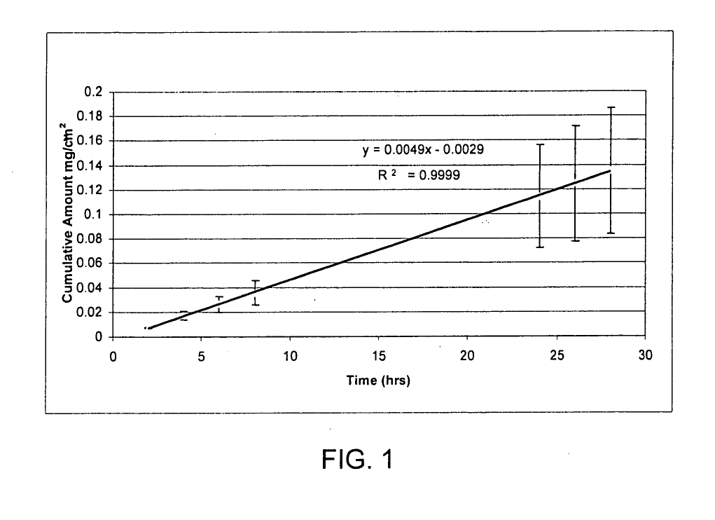

FIG. 2 is a graphical representation of the cumulative amount of ropivacaine delivered transdermally across human cadaver skin over time from a solidified gel formulation with similar composition in accordance with embodiments of the present invention, where steady-state delivery is shown over 30 hours.

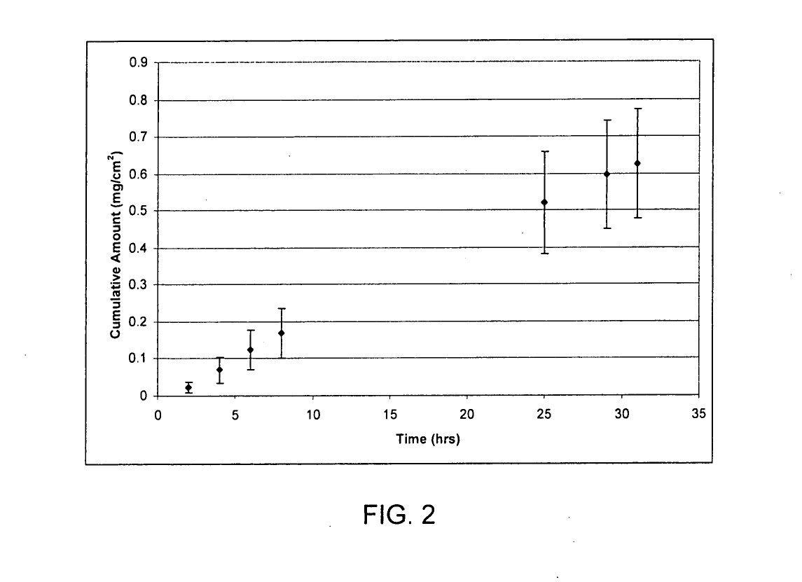

### DETAILED DESCRIPTION OF THE PREFERRED EMBODIMENTS

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions. “Skin” is defined to include human skin, finger and toe nail surfaces, and mucosal surfaces that are usually at least partially exposed to air such as lips, genital and anal mucosa, and nasal and oral mucosa.

The phrase “effective amount,” “therapeutically effective amount,” or “therapeutically effective rate(s)” of a drug refers to non-toxic, but sufficient amounts or delivery rates of a drug which achieves therapeutic results in treating a condition for which the drug is being delivered. It is understood that various biological factors may affect the ability of a substance to perform its intended task. Therefore, an “effective amount,” “therapeutically effective amount,” or “therapeutically effective rate(s)” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

The phrases “dermal drug delivery” or “dermal delivery of drugs” shall include both transdermal and topical drug delivery, and shall mean the delivery of drug(s) to, through, or into the skin. Transdermal delivery of drug can be targeted to skin tissues just under the skin, regional tissues or organs under the skin, systemic circulation, and/or the central nervous system.

The terms “flux,” “transdermal flux,” or “dermal flux” refer to the quantity of the drug permeated into or across skin per unit area per unit time. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver or freshly separated skin tissue from hairless mice rather than measuring drug flux across the skin using human volunteers, it is generally accepted by those skilled in the art that results from a properly designed and executed in vitro test can be used to estimate or predict the results of an in vivo test with reasonable reliability. Therefore, “flux” values referenced in this patent application can mean those measured by either in vivo or in vitro methods.

The term “drug(s)” refers to any bioactive agent that is applied to, into, or through the skin which is applied so as to achieve a therapeutic affect. This includes compositions that are traditionally identified as drugs, as well other bioactive agents that are not always considered to be “drugs” in the classic sense, e.g., peroxides, humectants, emollients, etc., but which can provide a therapeutic effect for certain conditions.

The term “drug form” refers to all possible chemical and/or physical forms of a drug. Examples of various drug forms include but are not limited to polymorphs, salts, hydrates, solvates, and cocrystals. For some drugs, one form of the drug may possess better physical-chemical properties making it more amenable for being delivered to, into, or through the skin, and this particular form is defined as the “physical form favorable for dermal delivery.” For example the steady state flux of diclofenac sodium from flux enabling non-volatile solvents is much higher than the steady state flux of diclofenac acid from the same flux enabling non-volatile solvents (compare Tables 10 and 11 below). It is therefore desirable to evaluate the flux of the physical forms of a drug from non-volatile solvents to select a desirable physical form/non-volatile solvent combination.

The term “flux-enabling non-volatile solvent” refers to a solvent or solvents selected specifically for a particular drug(s) and/or drug form. The solvent is non-volatile (less volatile than water) and, when containing saturated concentrations of the selected drug (and nothing else), can deliver a “therapeutically sufficient flux” of the selected drug across intact skin. There can be more than one flux enabling non-volatile solvent for any given drug. At saturated levels, though not required in the gel-forming formulations of the present invention, a solvent can be tested to determine whether it is a flux-enabling non-volatile solvent. Testing using this saturated drug-in-solvent state can be used to measure the maximum flux-generating ability of a non-volatile solvent system. To determine flux, the drug solvent mixture should be kept on the skin for a clinically sufficient amount of time. In reality, it is difficult to keep a solvent on the skin of a human volunteer for an extended period of time. Therefore, an alternative method to determine whether a solvent is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations.

There are generally two different ways to formulate a non-volatile solvent system that is “flux-enabling”: One approach is to optimize the permeation driving force for the drug (i.e., optimizing the solute activity coefficient in the formulation through selecting and testing various solvents and solvent mixtures, adjusting pH, different drug forms, etc.). A second approach is to use a chemical permeation enhancer(s) that reversibly alters the structure and hence the barrier properties of the skin to reach an otherwise unattainable therapeutic permeation rate. Although a non-volatile solvent system may be “flux-enabling” due to the combination of the two mechanisms, usually one of the mechanisms is dominantly responsible for the good permeability. There are several ways to tell which mechanism is dominant. For example, skin structure alteration using chemical permeation enhancers usually induces skin irritation, the magnitude of the irritation response being proportional to the degree of skin alteration. Therefore if the permeability of the drug increases proportionally with increasing concentration of a particular ingredient of the formulation, and additionally the increase in permeation is also accompanied with increasing skin irritation, the mechanism is predominantly a change in the skin structure.

Another method of determining which mechanism is dominant is to look at skin irritation. Significant skin irritation is a good indication that the mechanism is predominantly a skin structure change. In contrast, the optimization of permeation driving force usually involves low or no skin irritation. If the good permeability is due to optimization of permeation driving force, the maximum flux value is attained when a particular solvent(s) concentration is in a certain narrow range (as opposed to increasing monotonically with increasing concentrations of the ingredient(s)). This is clearly illustrated by the experimental data in Example 6 below: transdermal flux of clobetasol propionate in pure propylene glycol and pure isotearic acid is 3.8 and 19.4 mcg/cm²/hr, respectively, while in 9:1 propylene glycol: isostearic acid solution the flux was 764.7 mcg/cm²/hr.

If one disregards the issue of skin irritation, one can always add enough permeation enhancer(s) into a formulation to achieve desired permeability. On the other hand, optimizing permeation driving force typically requires more research effort and often involves experimenting with various solvents in different ratios, as well adjusting parameters such as lipophilicity/hydrophilicity, pH, etc. However, since skin irritation is a serious side effect, using optimization of permeation driving force to achieve desired permeability is a more preferred approach. In this patent application, unless otherwise specified, “flux enabling” is defined as that caused mainly by optimizing the permeation driving force with minimal or no skin structural change (low or no skin irritation). Although permeation enhancers are not required for the practice of the present invention, they can be included in the formulations in non-irritating amounts. “Therapeutically sufficient flux” is defined as the permeation flux of the selected drug that delivers sufficient amount of drug into or across the skin to be clinically beneficial. “Clinically beneficial,” when referring to flux, means that at least a portion of the patient population can obtain some degree of benefit from the drug flux. It does not necessarily mean that the majority of the patient population can obtain some degree of benefit or the benefit is high enough to be deemed “effective” by relevant government agencies or the medical profession. Therefore, “clinically beneficial” flux may be lower than “clinically effective” flux. More specifically, for drugs that target skin or regional tissues or organs close to the skin surface (such as joints, certain muscles, or tissues/organs that are at least partially within 5 cm of the skin surface), “therapeutically sufficient flux” refers to the drug flux that can deliver a sufficient amount of the drug into the target tissues within a clinically reasonable amount of time. For drugs that target the systemic circulation, “therapeutically sufficient flux” refers to drug flux that, via clinically reasonable skin contact area, can deliver sufficient amounts of the selected drug to generate clinically beneficial plasma or blood drug concentrations within a clinically reasonable time. Clinically reasonable skin contact area is defined as a size of skin application area that most patients would accept. Typically, a skin contact area of 400 cm² or less is considered reasonable. Therefore, in order to deliver 4000 μg of a drug to the systemic circulation via a 400 cm² skin contact area over 10 hours, the flux needs to be at least 4000 μg/400 cm²/10 hour, which equals 1 μg/cm²/hr. By this definition, different drugs have different therapeutically sufficient fluxes.

The following are estimates of “therapeutically sufficient flux” for some drugs:

TABLE 1
In vitro steady state flux values of various drugs

| | | Estimated |
|---|---|---|
| | | Therapeutically |
| | | sufficient flux* |
| Drug | Indication | (μg/cm²/h) |
| Ropivacaine** | Neuropathic pain | 5 |
| Lidocaine | Neuropathic pain | 30 |
| Acyclovir | Herpes simplex virus | 3 |
| Ketoprofen | Musculoskeletal pain | 16 |
| Diclofenac | Musculoskeletal pain | 1 |
| Clobetasol | Dermatitis, psoriasis, | 0.05 |
| | eczema | |
| Betamethasone | Dermatitis, psoriasis, | 0.01 |
| | eczema | |
| Testosterone | Hypogonadal men, | 0.8 |
| | hormone treatment for | |
| | postmenopausal women | |
| Imiquimod | Warts, basal cell | 0.2 |
| | carcinoma | |

*Flux determined using an in vitro method described in Example 1.
**Estimated flux based on known potency relative to lidocaine.

The therapeutically sufficient flux values in Table 1 (with the exception of ropivacaine) represent the steady state flux values of marketed products through hairless mouse or human epidermal membrane in an in vitro system described in Example 1. These values are meant only to be estimates and to provide a basis of comparison for formulation development and optimization.

The therapeutically sufficient flux for a selected drug could be very different for different diseases to be treated for, different stages of diseases, and different individual patients.

The following examples, listed in Table 2, illustrate selection of flux-enabling non-volatile solvents for some of the drugs specifically studied.

Experiments were carried out as described in Example 1 below and the results are further discussed in the subsequent Examples 2-9.

TABLE 2
In vitro steady state flux values of various drugs
from non-volatile solvent systems

| | | Average Flux* |
|---|---|---|
| Drug | Non-Volatile Solvent | (μg/cm2/hr) |
| Betamethasone | Oleic acid | 0.009 ± 0.003 |
| dipropionate | Sorbitan monolaurate | 0.03 ± 0.02 |
| Cobetasol propionate | Propylene glycol | 0.0038 ± 0.0004 |
| | Light mineral oil | 0.031 ± 0.003 |
| | Isostearic acid (ISA) | 0.019 ± 0.003 |
| Ropivacaine | Glycerol | 1.2 ± 0.7 |
| | Mineral oil | 8.9 ± 0.6 |
| Ketoprofen | Polyethylene glycol 400 | 5 ± 2 |
| | Span 20 | 15 ± 3 |
| Acyclovir | Polyethylene glycol 400 | 0 |
| | Isostearic acid + 10% | 2.7 ± 0.6 |
| | trolamine | |

*Each value represents the mean and st. dev of three determinations.

The in vitro steady state flux values in Table 2 from non-volatile solvents show surprising flux-enabling and non flux-enabling solvents. This information can be used to guide formulation development.

The term “flux-enabling, plasticizing non-volatile solvent” is defined as a flux-enabling non-volatile solvent that also has plasticizing effect on selected gel-forming agents. For example, propylene glycol is a “flux-enabling, plasticizing non-volatile solvent” for ketoprofen with polyvinyl alcohol as the selected gel-forming agent. However, the formulation containing propylene glycol as the “flux-enabling, plasticizing non-volatile solvent” for ketoprofen with Gantrez 97 or Avalure UR 405 as the gel-forming agent do not have the same plasticizing effect. The combination of propylene glycol and Gantrez 97 or Avalure UR 405 is less compatible and results in a less desirable formulation for topical applications.

Different drugs often have different flux-enabling non-volatile solvent systems which provide particularly good results. Examples of such are noted in Table 3. Experiments were carried out as described in Example 1 below and the results are further discussed in the subsequent Examples 2-9.

TABLE 3
In vitro steady state flux values of various drugs from particularly high
flux-enabling non-volatile solvent systems.

| | High flux-enabling | |
|---|---|---|
| | non-volatile | Avg. Flux* |
| Drug | solvent | (μg/cm2/h) |
| Ropivacaine | ISA | 11 ± 2 |
| | Span 20 | 26 ± 8 |
| Ketoprofen | Propylene glycol | 90 ± 50 |
| Acycolvir | ISA + 30% trolamine | 7 ± 2 |
| Betamethasone dipropionate | Propylene Glycol | 0.20 ± 0.07 |
| Clobetasol propionate | PG + ISA (Ratio of | 0.8 ± 0.2 |
| | PG:ISA ranging | |
| | from 200:1 to 1:1) | |

*Each value represents the mean and st. dev of three determinations.

It should be noted that “flux-enabling non-volatile solvent,” “flux-enabling, plasticizing non-volatile solvent,” or “high flux-enabling non-volatile solvent” can be a single chemical substance or a mixture of two or more chemical substances. For example, the steady state flux value for clobetasol propionate in Table 3 is a 9:1 for propylene glycol:isostearic acid mixture that generated much higher clobetasol flux than propylene glycol or ISA alone (see Table 2).

Therefore, the 9:1 propylene glycol:isostearic acid mixture is a “high flux-enabling non-volatile solvent” but propylene glycol or isostearic acid alone is not.

The phrase “substantially constant” when referring to “sustained delivery” of drug can be defined in terms of either an in vitro permeability across human or hairless mouse skin or epidermis, or by a data collected from a pool of 12 or more human subjects, wherein the drop in mean drug delivery rate over a specified period of time (about 2 hours or longer) is not more than 50% from a peak drug delivery rate. Thus, compositions that are delivered at a “substantially constant” rate include formulations that deliver a drug at substantially constant and therapeutically significant rates for a sustained period of time, e.g., at least about 2 hours, at least about 4 hours, at least about 8 hours, at least about 12 hours, at least about 24 hours, etc.

“Volatile solvent system” can be a single solvent or a mixture of solvents that are volatile, including water and solvents that are more volatile than water. Non-limiting examples of volatile solvents that can be used in the present invention include iso-amyl acetate, denatured alcohol, methanol, ethanol, isopropyl alcohol, propanol, C4-C6 hydrocarbons, butane, isobutene, pentane, hexane, acetone, water, chlorobutanol, ethyl acetate, fluro-chloro-hydrocarbons, turpentine, cytopentasiloxane, cyclomethicone, methyl ethyl ketone, other lower alcohols (containing 4 or less carbons) and mixtures thereof.

“Non-volatile solvent system” can be a single solvent or mixture of solvents that are less volatile than water. It can also contain substances that are solid or liquid at room temperatures, such as pH or ion-pairing agents. After evaporation of the volatile solvent system, most of the non-volatile solvent system should remain in the solidified layer for a period of time sufficient to adequately dermally delivery a given drug to, into, or through the skin of a subject at a sufficient flux for a period of time to provide a therapeutic effect. In some embodiments, in order to obtain desired permeability for an active drug and/or compatibility with gel-forming agents or other ingredients of the formulation, a mixture of two or more non-volatile solvents can be used to form the non-volatile solvent system. The non-volatile solvent system may also serve as a plasticizer of the solidified gel, so that the gel is elastic and flexible.

The term “solvent vehicle” describes compositions that include both a volatile solvent system and non-volatile solvent system. The volatile solvent system is chosen so as to evaporate from the adhesive gel forming formulation quickly to form a solidified layer, and the non-volatile solvent system is formulated or chosen to substantially remain as part of the solidified layer after volatile solvent system evaporation so as to provide continued delivery of the drug. Typically, the drug can be partially or completely dissolved in the solvent vehicle or formulation as a whole. Likewise, the drug can also be partially or completely solubilizable in the non-volatile solvent system once the volatile solvent system is evaporated. Formulations in which the drug is only partially dissolved in the non-volatile solvent system after the evaporation of the volatile solvent system have the potential to maintain longer duration of sustained delivery, as the undissolved drug can dissolve into the non-volatile solvent system as the dissolved drug is depleted from the solidified layer during drug delivery.

The term “sustained period of time” is defined as at least 30 minutes, preferably at least about 2 hours, and often at least about 8 hours, 24 hours, 72 hours, or more.

“Adhesive gel forming formulation”, “gel forming formulation”, or “adhesive solid gel-forming formulation” refer to a composition that has a viscosity suitable for application to a skin surface prior to evaporation of its volatile solvent(s), and which can become a solidified (or solid gel) layer after evaporation of at least a portion of the volatile solvent(s). The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

The terms “washable” or “removed by washing” when used with respect to the adhesive gel forming formulations of the present invention refers to the ability of the adhesive gel forming formulation to be removed by the application of a washing solvent using a normal or medium amount of washing force. The required force to remove the gel forming formulations by washing should not cause significant skin irritation or abrasion. Generally, gentle washing force accompanied by the application of an appropriate washing solvent is sufficient to remove the adhesive gel forming formulations disclosed herein. The solvents which can be used for removing by washing the gel forming formulations of the present invention are numerous, but preferably are chosen from commonly acceptable solvents including the volatile solvents listed herein. Preferred washing solvents do not significantly irritate human skin and are generally available to the average subject. Examples of preferred washing solvents include but are not limited to water, ethanol, isopropyl alcohol, methanol, propanol, acetone, and ethyl acetate. Surfactants can also be used in some embodiments.

The term “drying time” or “acceptable length of time” refer to the time it takes for the formulation to form a non-messy solidified surface after application on skin under standard skin and ambient conditions, and with standard testing procedure. It is noted that the word “drying time” in this application does not mean the time it takes to completely evaporate off the volatile solvent(s). Instead, it means the time it takes to form the non-messy solidified surface as described above.

The term “non-messy” when used to describe the solidified gels of the present invention, in particular the exterior surfaces (the surfaces not in contact with the skin) refers to the coherent nature of the solidified gel. When an acceptable drying time has passed, the gel, in particular the exterior surface of the gel, become coherent such that the exterior surface does not readily lose mass when contacted with other surfaces, e.g., clothing, etc.

“Standard skin” or “normal skin” is defined as dry, healthy human skin having a surface temperature of between 32° C. to 36° C. Standard ambient conditions are defined by the temperature range of from 20° C. to 25° C. and a relative humidity range of from 20% to 80%.

The “standard testing procedure” or “standard testing condition” is as follows: To standard skin at standard ambient conditions is applied an approximately 0.2 mm layer of the adhesive gel-forming formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer”, “dried gel layer”, “dried layer”, “solid gel layer” or similar phrases, used interchangeably, describe the solidified or dried layer of an adhesive solid gel-forming formulation after at least a portion of the volatile solvent system has evaporated. The solidified layer remains adhered to the skin, and is preferably capable of maintaining good contact with the patient's skin for substantially the entire duration of application under normal skin and ambient conditions. A solidified gel layer can be a layer of a solid gel-forming formulation that forms after sufficient amount of the volatile solvent(s) have evaporated so that a non-messy surface of the layer remains on the top, but the formulation underneath the non-messy surface is still not solidified yet. In other words, a solidified gel layer is defined to include only partially solidified layer. The solidified layer may be peeled off the skin or washed off with solvent, such as water or ethanol, at the end of the desired drug delivery. Other solvents which could also be used to wash off the solidified gel formulation include but are not limited to the volatile solvents listed herein. For certain formulations, applications and/or individuals, the solidified layer is better removed by peeling off. For others, the solidified layer is better removed by washing off with a solvent. For example, if the solid-gel-forming formulation is applied to a body area with a lot of hair (e.g. an anti genital herpes solid gel-forming formulation applied on genital skin area with pubic hair), removal by peeling might cause discomfort and therefore be undesirable. In another example, if the solid-gel-forming formulation is applied to a palmar surface, such as the palm of the hand or the sole of a foot, the ability for removal by peeling may be secondary consideration to a formulation that will adhere to the skin surface. In these cases, a solidified gel layer configured to be easily washed off by water or ethanol may be more desirable. In washing embodiments, the solvent used to wash off the solidified gel layer may dissolve the layer or make it less adhesive to the skin so that it can be easily removed from the skin.

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, the present invention is drawn to an adhesive solid gel-forming formulation for dermal delivery of a drug can comprise a drug, a solvent vehicle, and a gelling agent. The solvent vehicle can comprise a volatile solvent system having one or more volatile solvent(s) and a non-volatile solvent system having one or more non-volatile solvent(s), wherein the non-volatile solvent system comprises at least one flux-enabling non-volatile solvent for the drug such that the drug can be delivered in therapeutically effective amounts over a period of time, even after most of the volatile solvent(s) is evaporated. The formulation can have viscosity suitable for application to the skin surface prior to evaporation of at least one volatile solvent, and can further be configured such that when applied to the skin surface, the formulation forms a solidified gel layer after at least a portion of the volatile solvent(s) is evaporated.

In an alternative embodiment, a method of dermally delivering a drug to, into, or through the skin can comprise applying an adhesive solid gel-forming formulation to a skin surface of the subject, dermally delivering the drug from the solidified gel layer over a period of time and at desired rates, and removing the solidified gel layer from the skin after a period of time has elapsed or the desired quantity of the drug has been delivered. Removal of the solid gel formulation can be done by washing with solvents or peeling. The adhesive solid gel-forming formulation can include a drug, a solvent vehicle, and a gelling agent. The solvent vehicle can comprise a volatile solvent system having one or more volatile solvent(s) and a non-volatile solvent system having one or more non-volatile solvent(s), wherein at least one of the non-volatile solvent or the mixture of non-volatile solvents is flux-enabling. The formulation can have a viscosity suitable for application to a skin surface prior to evaporation of the volatile solvent. When the formulation is applied to the skin surface, the formulation can form a solidified gel layer after at least a portion of the volatile solvent system evaporated.

In another embodiment, a method of preparing an adhesive solidified gel formulation for dermal drug delivery can comprise steps of selecting a drug suitable for dermal delivery; selecting or formulating a non-volatile solvent or a mixture of non-volatile solvents that is flux-enabling for the selected drug, selecting a gelling agent that is compatible with the drug and the non-volatile solvent, selecting or formulating a volatile solvent system that is compatible with the drug, the non-volatile solvent and the gelling agent; and formulating all above ingredients into an adhesive solidified gel-forming formulation. The adhesive solid gel-forming formulation can have a viscosity suitable for application to a skin surface prior to evaporation of the volatile solvent system, and can be applied to the skin surface where it forms a solidified gel layer after at least a portion of the volatile solvent system is evaporated. In this embodiment, the drug continues to be delivered at a therapeutically effective amount after the volatile solvent system is substantially evaporated.

In still another embodiment, a solidified gel layer for delivering a drug can comprise a drug, a non-volatile solvent system, and a gelling agent. The non-volatile solvent system can include at least one flux-enabling non-volatile solvent or a mixture of non-volatile solvents that are flux-enabling. Further, the solidified gel layer can be stretched in at least one direction by 5%, or even 10%, without breaking, cracking, or separation from a skin surface to which the solidified gel layer is applied.

Thus, these embodiments exemplify the present invention which is related to novel formulations, methods, and solidified gel layers that are typically in the initial form of semi-solids (including creams, gels, pastes, ointments, and other viscous liquids), which can be easily applied onto the skin as a layer, and can quickly (from 15 seconds to about 4 minutes under normal skin and ambient conditions) to moderately quickly (from about 4 to about 15 minutes under normal skin and ambient conditions) change into a solidified gel layer for drug delivery. A solidified gel layer thus formed is capable of delivering drug to the skin, into the skin, across the skin, etc., at substantially constant rates, over an sustained period of time, e.g., hours to tens of hours, so that most of the active drug is delivered after the solidified gel layer is formed.

Additionally, the solidified gel layer typically adheres to the skin, but has a solidified, minimally-adhering, outer surface which is formed relatively soon after application and which does not substantially transfer to or otherwise soil clothing or other objects that a subject is wearing or that the solidified gel layer may inadvertently contact. The solidified gel layer can also be formulated such that it is highly flexible and stretchable, and thus capable of maintaining good contact with a skin surface, even if the skin is stretched during body movement, such as at a knee, finger, elbow, or other joints.

In selecting the various components that can be used, e.g., drug, solvent vehicle of volatile solvent system and non-volatile solvent system, gelling agent(s), etc., various considerations can occur. For example, the volatile solvent system can be selected from pharmaceutically or cosmetically acceptable solvents known in the art. Examples of such volatile solvents include but are not limited to iso-amyl acetate, denatured alcohol, methanol, ethanol, isopropyl alcohol, propanol, C4-C6 hydrocarbons, butane, isobutene, pentane, hexane, acetone, water, chlorobutanol, ethyl acetate, fluro-chloro-hydrocarbons, turpentine, cytopentasiloxane, cyclomethicone, methyl ethyl ketone, ethyl ether, mixtures thereof, and mixtures with water thereof. Additionally, these volatile solvents should be chosen to be compatible with the rest of the formulation. It is desirable to use an appropriate weight percentage of the volatile solvent(s) in the formulation. Too much of the volatile solvent system prolongs the drying time. Too little of the volatile solvent system can make it difficult to spread the formulation on the skin. For most formulations, the weight percentage of the volatile solvent(s) can be from about 2 wt % to about 50 wt %, and more preferably from about 4 wt % to about 30 wt %.

The volatile solvent system can also be chosen to be compatible with the non-volatile solvent, gelling agent, drug, and any other excipients that may be present. For example, polyvinyl alcohol (PVA) is not soluble in ethanol. Therefore, a volatile solvent which will dissolve PVA needs to be formulated in the solidified gel. For instance, water will dissolve PVA and can be utilized as a volatile solvent in a solid-gel forming formulation; however the drying time in such a formulation may be too long to certain applications. Therefore, a second volatile solvent (e.g., ethanol) can be formulated into the formulation to reduce the water content but maintain a sufficient amount of water to keep PVA in solution and thereby reduce the drying time.

The non-volatile solvent system can also be chosen or formulated to be compatible with the gelling agent, the drug, the volatile solvent, and any other ingredients that may be present. For example, the gelling agent can be chosen so that it is dispersible or soluble in the non-volatile solvent system. Most non-volatile solvent systems and solvent vehicles as a whole will be formulated appropriately after experimentation. For instance, certain drugs have good solubility in poly ethylene glycol (PEG) having a molecular weight of 400 (PEG 400, non-volatile solvent) but poor solubility in glycerol (non-volatile solvent) and water (volatile solvent). However, PEG 400 cannot effectively dissolve poly vinyl alcohol (PVA), and thus, is not very compatible alone with PVA, a gelling agent. In order to dissolve sufficient amount of an active drug and use PVA as a gelling agent at the same time, a non-solvent system including PEG 400 and glycerol (compatible with PVA) in an appropriate ratio can be formulated, achieving a compatibility compromise. As a further example of compatibility, non-volatile solvent/gelling agent incompatibility is observed when Span 20 (sorbitan laurate) is formulated into a gel formulation containing PVA. With this combination, Span 20 can separate out of the formulation and form an oily layer on the surface of the solidified gel layer. Thus, appropriate gelling agent/non-volatile solvent selections are desirable in developing a viable formulation and compatible combinations.

In further detail, non-volatile solvent(s) that can be used alone or in combination to form non-volatile solvent systems can be selected from a variety of pharmaceutically acceptable liquids, including but not limited to 1,2,6-hexanetriol, alkyltriols, alkyldiols, acetyl monoglycerides, tocopherols, alkyl dioxolanes, p-propenylanisole, dimethyl isosorbide, alkyl glucoside, benzoic acid, benzyl alcohol, butyl alcohol, beeswax, benzyl benzoate, butylene glycol, caprylic/capric triglyceride, caramel, cinnamaldehyde, cocoa butter, cocoglycerides, corn syrup, cresol, cyclomethicone, diacetin, diacetylated monoglycerides, dibutyl sebecate, diethanolamine, diethylene glycol monoethyl ether, diglycerides, dipropylene glycol, ethylene glycol, eugenol, fat, fatty acid (esters glycerides), fatty alcohols, liquid sugars, ginger extract, glycerin, high fructose corn syrup, IPM, IP palmitate, isostearic acidlimonene, milk, mineral oil, monoacetin, monoglycerides, oleic acid, octyldodecanol, oleyl alcohol, PEG (propylene glycols), vegetable oils including, olive alcohol, palm oil, corn oil, cottonseed oil, cinnamon oil, clove oil, coconut oil, anise oil, apricot oil, coriander oil, cassia oil, castor oil, lemon oil, lime oil, pine needle oil, sesame oil, spearmint oil, soybean oil, eucalyptus oil, hydrogenated castor oil, orange oil, nutmeg oil, peanut oil, peppermint oil, petrolatum, phenol, polypropylene glycol, propylene glycol, trolamine, tromethemine, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters including PEG-stearates, PEG-oleates, PEG-laurates, PEG fatty acid diesters including PEG-dioleates, PEG-distearates, PEG-castor oils, glyceryl behenate, PEG glycerol fatty acid esters including PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid multisterol extract, myristyl alcohol, neutral oil, PEG-octyl phenyl ethers, PEG-alkyl ethers including PEG-cetyl ethers, PEG-stearyl ethers, PEG-sorbitan fatty acid esters including PEG-sorbitan diisosterates, PEG-sorbitan monostearates, propylene glycol fatty acid esters including propylene glycol stearates, propylene glycol caprylate/caprates, sodium pyrrolidone carboxylate, sorbitol, squalene, stear-o-wet, triacetin, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty alcohol ethers, alkyl-amides (N,N-dimethylalkylamides), N-methyl pyrrolidone related compounds, sorbitan fatty acid surfactants including sorbitan monooleate, sorbitan trioleate, sorbitan monopalmitate, ethyl oleate, polyglycerized fatty acids, glycerol monooleate, glyceryl monomyristate, glycerol esters of fatty acids, and mixtures thereof.

In addition to these and other considerations, the non-volatile solvent system can also serve as plasticizer in the solid-gel forming formulation so that when the solidified gel layer is formed, the layer is flexible, stretchable, and/or otherwise “skin friendly.”

Certain volatile and/or nonvolatile solvent(s) that are irritating to the skin may be desirable to use to achieve the desired solubility and/or permeability of the drug. It is also desirable to add compounds that are both capable of preventing or reducing skin irritation and are compatible with the formulation. For example, in a formulation where the volatile solvent is capable of irritating the skin, it would be helpful to use a non-volatile solvent that is capable of reducing skin irritation. Examples of solvents that are known to be capable of preventing or reducing skin irritation include, but are not limited to, glycerin, honey, and propylene glycol.

The formulations of the current invention may also contain two or more non-volatile solvents that independently are not flux-enabling non-volatile solvents for a drug but when formulated together become a flux enabling non-volatile solvent system. One possible reason for these initially non-flux enabling non-volatile solvents to become flux enabling non-volatile solvents when formulated together may be due to the optimization of the ionization state of the drug to a physical form which has higher flux or the non-volatile solvents act in some other synergistic manner. One further benefit of the mixing of the non-volatile solvents is that it may optimize the pH of the formulation or the skin tissues under the formulation layer to minimize irritation. Examples of suitable combinations of non-volatile solvents that result in an adequate non-volatile solvent system include but are not limited to isostearic acid/trolamine, isostearic acid/diisopropyl amine, oleic acid/trolamine, and propylene glycol/isostearic acid. Sometimes, however, two or more non-volatile solvents that individually are are not flux-enabling non-volatile solvents for a particular drug, can act as flux-enabling solvents when formulated together. Such combinations are included within the scope of the current invention.

The selection of the gelling agent can also be carried out in consideration of the other components present in the adhesive solid gel forming formulation. The gelling agent can be selected or formulated to be compatible to the drug and the solvent vehicle (including the volatile solvent(s) and the non-volatile solvent system), as well as to provide desired physical properties to the solidified gel layer once it is formed. Depending on the drug, solvent vehicle, and/or other components that may be present, the gelling agent can be selected from a variety of agents, including but not limited to polyethylene oxide, ammonia methacrylate, carrageenan, cellulose acetate phthalate aqueous such as CAPNF from Eastman, carboxy methyl cellulose Na, carboxy polymethylene, cellulose, cellulose acetate (microcrystalline), cellulose polymers, divinyl benzene styrene, ethyl cellulose, ethylene vinyl acetate, silicone, polyisobutylene, shellac (FMC BioPolymer), guar gum, guar rosin, cellulose derivatives such as hydroxy ethyl cellulose, hydroxy methyl cellulose, hydroxy propyl cellulose, hydroxypropyl methyl cellulose, carboxymethyl cellulose, and methyl cellulose, hypromellose phthalate (hydroxypropyl methylcellulose phthalate), methyl acrylate, microcrystalline wax, polyvinyl alcohol, polyvinyl acetate, polyvinyl acetate phthalate such as Suretic from Colorcon, PVP ethyl cellulose, polyvinyl yrrolidone (PVP), acrylate, PEG/PVP, xantham Gum, trimethyl siloxysilicate, maleic acid/anhydride copolymersl, polacrilin, poloxamer, polyethylene oxide, poly glactic acid /poly-l-lactic acid, turpene resin, locust bean gum, prolamine (Zein), acrylic copolymers, polyurethane dispersions, gelatin (both type A and type B from various sources such as pig, cattle, and fish),dextrin, starch, polyvinyl alcohol-polyethylene glycol copolymers, methyacrylic acid-ethyl acrylate copolymers such as BASF's Kollicoat polymers, methacrylic acid and methacrylate based polymers such as poly(methacrylic acid) copolymers and methylmethacrylate copolymers, including Rohm and Haas' Eudragit polymers (Eudragit (E, L, NE, RL, RS, S100)), Esters of polyvinylmethylether/maleic anhydride copolymer such as Gantrez ES-425, Gantrez ES-225 available from ISP, and mixtures thereof. Other polymers may also be suitable as the solid gel-forming agent, depending on the solvent vehicle components, the drug, and the specific functional requirements of the given formulation.

In one embodiment, the non-volatile solvent system and the gelling agent(s) should be compatible with each other. Compatibility can be defined as i) the gelling agent does not substantially negatively influence the function of the non-volatile solvent system, except for some acceptable reduction of flux; ii) the gelling agent can hold the non-volatile solvent system in the solidified gel layer so that substantially no non-volatile solvent oozes out of the layer, and/or iii) the solidified gel layer formed with the selected non-volatile solvent system and the gelling agent has acceptable flexibility, rigidity, tensile strength, elasticity, and adhesiveness. The weight ratio of the non-volatile solvent system to the gelling agent(s) can be from about 0.01:1 to about 10:1. In another aspect, the ratio between the non-volatile solvent system and the gelling agent can be from about 0.2:1 to about 4:1. In yet another aspect, the weight ratio between the non-volatile solvent system and the gelling agent can be from about 0.6:1 to about 1.5:1.

The thickness of the formulation layer applied on the skin should also be appropriate for a given formulation and desired drug delivery considerations. If the layer is too thin, the amount of the drug may not be sufficient to support sustained delivery over the desired length of time. If the layer is too thick, it may take too long to form a non-messy exterior surface of the solidified gel layer. If the drug is very potent and the solidified gel has very high tensile strength, a layer as thin as 0.01 mm may be sufficient. If the drug has rather low potency and the solidified gel has low tensile strength, a layer as thick as 2-3 mm may be desirable. Thus, for most drugs and formulations, the appropriate thickness can be from about 0.01 mm to about 3 mm, but more typically, from about 0.05 mm to about 1 mm.

The flexibility and stretchability of a solidified gel layer can be desirable in some applications. For instance, certain non-steroidal anti-inflammatory agents (NSAIDs) can be applied directly over joints and muscles for transdermal delivery to joints and muscles. However, skin areas over joints and certain muscle groups are often significantly stretched during body movements. Such movement prevents non-stretchable patches from maintaining good skin contact. Lotions, ointments, creams, gels, foams, pastes, or the like also may not be suitable for use for the reasons cited above. As such, in transdermal delivery of NSAIDs into joints and/or muscles, the solid gel-forming formulations of the present invention can offer unique advantages and benefits. It should be pointed out that although good stretchability can be desirable in some applications, the solid gel-forming formulations of the present invention do not always need to be stretchable, as certain applications of the present invention do not necessarily benefit from this property. For instance, if the formulation is applied on a small facial area overnight for treating acne, a patient would experience minimal discomfort and formulation-skin separation even if the solidified gel layer is not stretchable, as facial skin usually is not stretched very much during a sleep cycle.

A further feature of a formulation prepared in accordance with embodiments of the present invention is related to drying time. If a formulation dries too quickly, the user may not have sufficient time to spread the formulation into a thin layer on the skin surface before the formulation is solidified, leading to poor skin contact. If the formulation dries too slowly, the patient may have to wait a long time before resuming normal activities (e.g. putting clothing on) that may remove un-solidified formulation. Thus, it is desirable for the drying time to be longer than about 15 seconds but shorter than about 15 minutes (under the “standard testing condition” as defined above), and preferably from about 0.5 minutes to about 6 minutes.

Other benefits of the solidified gel layers of the present invention include the presence of a physical barrier that can be formed by the material itself. For instance, local anesthetic agents and other agents such as clonidine may be delivered topically for treating pain related to neuropathy, such as diabetic neuropathic pain. Since many of such patients feel tremendous pain, even when their skin area is only gently touched, the physical barrier of the solidified gel layer can prevent or minimize pain caused by accidental contact with objects or others. In some circumstances, the physical barrier of the solid gel formation may also act to inhibit or prevent infection.

These and other advantage can be summarized in the following non-limiting list of benefits, as follows. The solidified gel layers of the present invention can be prepared in an initial form that is easy to apply as a semisolid dosage form. Additionally, upon volatile solvent system evaporation, the dosage form is relatively thick and can contain much more active drug than a typical layer of traditional cream, gel, lotion, ointment, paste, etc., and further, is not as subject to unintentional removal. Further, as the solidified gel layer remains adhesive to skin, easy removal of the solidified gel layer can be accomplished by peeling off or washing off with a solvent such as water or ethanol. In some embodiments, the adhesion to skin and elasticity of the material is such that the solidified gel layer will not separate from the skin upon skin stretching at highly stretchable skin areas, such as over joints and muscles. For example, in one embodiment, the solidified gel layer can be stretched by 5%, or even 10% or greater, in one direction without cracking, breaking, and/or separating form a skin surface to which the solidified gel layer is applied. Still further, the solidified gel layer can be configured to advantageously deliver drug and protect sensitive skin areas without cracking or breaking.

Specific examples of applications that can benefit from the systems, formulations, and methods of the present invention are as follows. In one embodiment, a solidified gel layer including bupivacaine, lidocaine, or ropivacaine, can be formulated for treating diabetic and post herpetic neuralgia. Alternatively, dibucanine and an alpha-2 agonist such as clonidine can be formulated in a solid gel forming formulation for treating the same disease. In another embodiment, retinoic acid and benzoyl peroxide can be combined in a solid gel forming formulation for treating acne, or alternatively, 1 wt % clindamycin and 5 wt % benzoyl peroxide can be combined in a formulation for treating acne. In another embodiment, a retinol solid gel-forming formulation (OTC) can be prepared for treating wrinkles, or a lidocaine solid gel-forming formulation can be prepared for treating back pain. In another embodiment, a zinc oxide solid gel-forming formulation (OTC) can be prepared for treating diaper rash, or an antihistamine solid gel-forming formulation can be prepared for treating allergic rashes such as poison ivy.

Additional applications include delivering drugs for treating certain skin conditions, e.g., dermatitis, psoriasis, eczema, skin cancer, viral infections such as cold sores and genital herpes infections, shingles, etc., particularly those that occur over joints or muscles where a transdermal patch may not be practical. For example, solid gel-forming formulations containing imiquimod can be formulated for treating skin cancer, common and genital warts, and actinic keratosis. Solid gel-forming formulations containing antiviral drugs such as acyclovir, penciclovir, famciclovir, valacyclovir, steroids, and behenyl alcohol can be formulated for treating herpes viral infections such as cold sores on the face or affected genital areas. Solid gel-forming formulations containing non-steroidal anti-inflammatory drugs (NSAIDs), capsaicin, alpha-2 agonists, and/or nerve growth factors can be formulated for treating soft tissue injury and muscle-skeletal pains such as joint and back pain of various causes. As discussed above, patches over these skin areas typically do not have good contact over sustained period of time, especially for a physically active patient, and may cause discomfort. Likewise, traditional semi-solid formulations such as creams, lotions, ointments, etc., may prematurely stop the delivery of a drug due to the evaporation of solvent and/or unintentional removal of the formulation. The solid gel-forming formulations of the present invention address the shortcomings of both of these types of delivery systems. In addition, because the gel-forming formulations of the present invention are washable they allow for easy and pain free removal of the gel from skin areas having hair.

One embodiment can entail a solid gel-forming formulation containing a drug from the class of alpha-2 antagonists which is applied topically to treat neuropathic pain. The alpha-2 agonist is gradually released from the formulation to provide pain relief over a sustained period of time. The surface of the formulation can become a coherent, soft solid after 2-4 minutes and the dried solid gel layer remains adhered to the body surface for the length of its application. The dried solid gel layer is easily removed after desired application time by peeling off or washing off with a solvent such as water, acetone or ethanol.

Another embodiment involves a solid gel-forming formulation containing capsaicin or a capsaicinoid which is applied topically to treat neuropathic pain. The capsaicin or capsaicinoid is gradually released from the formulation for treating this pain over a sustained period of time. The surface of the formulation can become a coherent, soft solid after 2-4 minutes and solidified solid gel layer remains adhered to the body surface for the length of its application. The dried solid gel layer is easily removed after desired application time by peeling off or washing off with a solvent such as water, acetone or ethanol.

Another embodiment involves solid gel-forming formulations containing tazorac for treating stretch marks, wrinkles, sebaceous hyperplasia, seborrheic keratosis. In another embodiment, solid gel-forming formulations containing glycerol can be made so as to provide a protective barrier for fissuring on finger tips.

Still another embodiment can include a solid gel-forming formulation containing a drug selected from the local anesthetic class such lidocaine and ropivacaine or the like, or NSAID class, such as ketoprofen, piroxicam, diclofenac, indomethacin, or the like, which is applied topically to treat symptoms of back pain, muscle tension, or myofascial pain or a combination thereof. The local anesthetic and/or NSAID is gradually released from the formulation to provide pain relief over a sustained period of time. The surface of the formulation layer can become a coherent, soft solid after about 2-4 minutes and the solidified gel layer remains adhered to the body surface for the length of its application. The dried solid gel layer is easily removed after desired application time by peeling off or washing off with a solvent such as water, acetone, or ethanol.

A further embodiment involves a solid gel-forming formulation containing at least one alpha-2 agonist drug, at least one tricyclic antidepressant agent, and/or at least one local anesthetic drug which is applied topically to treat neuropathic pain. The drugs are gradually released from the formulation to provide pain relief over a sustained period of time. The surface of the formulation layer can become a coherent, soft solid after 2-4 minutes and solidified gel layer remains adhered to the body surface for the length of its application. The dried solid gel layer is easily removed after desired application time by peeling off or washing off with a solvent such as water, acetone or ethanol.

A similar embodiment can include a solid gel-forming formulation containing capsaicin and a local anesthetic drug which is applied topically to the skin to provide pain relief. Another embodiment can include a solid gel-forming formulation containing the combination of a local anesthetic and a NSAID. In both of the above embodiments the drugs are gradually released from the formulation to provide pain relief over a sustained period of time. The surface of the formulation layer can become a coherent, soft solid after 2-4 minutes and solidified gel layer remains adhered to the body surface for the length of its application. The dried solid gel layer is easily removed after desired application time by peeling off or washing off with a solvent such as water, acetone, or ethanol.

In another embodiment, solid gel-forming formulations for the delivery of drugs that treat the causes or symptoms of diseases involving joints and muscles can also benefit from the systems, formulations, and methods of the present invention. Such diseases that may be applicable include, but not limited to, osteoarthritis (OA), rheumatoid arthritis (RA), joint and skeletal pain of various other causes, myofascial pain, muscular pain, and sports injuries. Drugs or drug classes that can be used for such applications include, but are not limited to, non-steroidal anti-inflammatory drugs (NSAIDs) such as ketoprofen and diclofanec, COX-2 selective NSAIDs and agents, COX-3 selective NSAIDs and agents, local anesthetics such as lidocaine, bupivacaine, ropivacaine, and tetracaine, and steroids such as dexamethasone.

Delivering drugs for the treatment of acne and other skin conditions can also benefit from principles of the present invention, especially when delivering drugs having low skin permeability. Currently, topical retinoids, peroxides, and antibiotics for treating acne are mostly applied as traditional semisolid gels or creams. However, due to the shortcomings as described above, sustained delivery over many hours is unlikely. For example, clindamycin, benzoyl peroxide, and erythromycin may be efficacious only if sufficient quantities are delivered into hair follicles. However, a traditional semisolid formulation, such as the popular acne medicine benzaclin gel, typically loses most of its solvent (water in the case of benzaclin) within a few minutes after the application. This short period of a few minutes likely substantially compromises the sustained delivery of the drug. The formulations of the present invention typically do not have this limitation.

In another embodiment, the delivery of drugs for treating neuropathic pain can also benefit from the methods, systems, and formulations of the present invention. A patch containing a local anesthetic agent, such as Lidoderm™, is widely used for treating neuropathic pain, such as pain caused by post-herpetic neuralgia and diabetes induced neuropathic pain. Due to the limitations of the patch as discussed above, the solidified gel layers prepared in accordance with the present invention provide some unique benefits including being a potentially less expensive alternative to the use of a patch. Possible drugs delivered for such applications include, but are not limited to, local anesthetics such as lidocaine, prilocaine, tetracaine, bupivicaine, etidocaine; and other drugs including capsaicin and alpha-2 agonists such as clonidine, dissociative anesthetics such as ketamine, tricyclic antidepressants such as amitriptyline.

In yet another embodiment, the delivery of medication for treating warts and other skin conditions would also benefit from long periods of sustained drug delivery. Such drugs that can be used in the formulations of the present invention include, but are not limited to, salicylic acid and imiquimod.

In another embodiment, the delivery of natural substances and nutrients such as retinol (Vitamin A) and humectants or emollients to the skin for cosmetic purposes can also benefit from the systems, formulations, and methods of the present invention.

A further embodiment involves controlled delivery of nicotine for treating nicotine dependence among smokers and persons addicted to nicotine. Formulations of the present invention would be a cost effective way of delivering therapeutic amounts of nicotine transdermally.

Another embodiment involves using the solid gel-forming formulation to deliver anti-histamine agents such as diphenhydramine, tripelennamine, fexofenadine desloratadine loratidine, cetirizine, and combinations thereof. These agents would reduce itching by blocking the histamine that causes the itch and also provide relief by providing topical analgesia.

A further embodiment involves the delivery of anti-fungal agents such as ciclopirox, imidazoles, miconazole, clotrimazole, econazole, ketoconazole, oxiconazole, sulconazole and allylamine derivatives such as butenafine, naftifine, fluconazole, terbinafine, and combinations thereof to the skin so as to eliminate or alleviate various fungal disorders such as nail fungal infections, athlete's foot and diaper rash. Delivery can be accomplished through the systems, formulations and methods of the present invention.

In another embodiment, delivery of antiviral agents such as acyclovir, trifluridine, idoxuridine, penciclovir, famciclovir, cidofovir, gancyclovir, valacyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, and combinations thereof. Anti-viral treatment could be used to treat both localized and systemic viral infections, such as cold sore or genital herpes.

A further embodiment involves the solid gel-forming formulations for the delivery of topically and systemically targeted anti-infectants such as antibiotics.

A further embodiment involves the solid gel-forming formulations for the delivery of sex steroids including the androgens, estrogens and progestagens such as testosterone, estradiol, progesterone, and other natural or synthetic male and female hormones. Examples of androgens which can be used in the formulations of the present invention include but are not limited to testosterone, methyl testosterone, oxandrolone, androstenedione, dihydrotestosterone, a pharmaceutically active derivative thereof, and combinations thereof. Non-limiting examples of estrogens and progesterone include estradiol, ethniyl estradiol, estiol, estrone, conjugated estrogens, esterified estrogens, estropipate, progesterone, norethindrone, norethindroneacetate, desogestrel, drospirenone, ethynodiol diacetate, norelgestromin, norgestimate, levonorgestrel, dl-norgestrel, cyproterone acetate, dydrogesterone, medroxyprogesterone acetate, chlormadinone acetate, megestrol, promegestone, norethisterone, lynestrenol, gestodene, tibolene, and combinations thereof.

A further embodiment involves the following steps: selecting a drug for dermal delivery, selecting or formulating a flux-enabling or high flux-enabling non-volatile solvent for the selected drug, selecting a gelling agent that is compatible with said flux-enabling or high flux-enabling non-volatile solvent and volatile solvent system, selecting a volatile solvent system that meets a preferred drying time frame and is compatible with the above ingredients, and formulating above ingredients into a solid gel-forming formulation that optionally further includes other ingredients such as viscosity modifying agent(s), pH modifying agent(s), and emollients.

Another embodiment involves a method of maintaining a liquid flux-enabling solvent on human skin, mucosa, or nail surfaces for delivery of a drug into tissues under said surfaces, comprising selecting a drug for dermal delivery, selecting or formulating a flux-enabling non-volatile solvent for the selected drug, selecting a gelling agent that is compatible with said flux-enabling non-volatile solvent and volatile solvent system, selecting a volatile solvent system, and formulating above ingredients into a solid gel-forming formulation.

Another embodiment involves a method for keeping a liquid flux-enabling non-volatile solvent on human skin for delivery of a drug into said human skin or tissues under said human skin. The method includes applying to a human skin a layer a formulation comprising a drug, a flux enabling non-volatile solvent, a gelling agent capable of gelling said liquid enabling non-volatile solvent into a soft solid, and a volatile solvent system that is compatible with the rest of components of the formulation. The formulation layer is such that, when it is applied to the skin, the evaporation of at least some of the volatile solvent system transforms the formulation from an initial less than solid state into a soft solid layer. The drug in the soft solid layer is delivered at therapeutically effective rates for a sustained period of time.

Other drugs that can be delivered using the formulations and methods of the current invention include humectants, emollients, and other skin care compounds.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Skin Permeation Methodology

Hairless mouse skin (HMS) or human epidermal membrane is used as the model membrane for the in vitro flux studies described in herein. Freshly separated epidermis removed from the abdomen of a hairless mouse or previously prepared human epidermal membrane samples are mounted carefully between the donor and receiver chambers of a Franz diffusion cell.

The receiver chamber is filled with pH 7.4 phosphate buffered saline (PBS).

The experiment is initiated by placing test formulations (of Examples 2-5) on the stratum corneum (SC) of the skin sample. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time. It is to be noted that human cadaver skin is used as the model membrane for the in vitro flux studies as indicated in some of the examples below. The mounting of the skin and the sampling techniques used are the same as described previously for the HMS studies.

### Example 2

Formulations of acyclovir (obtained from Uqufia) in various non-volatile solvent systems are evaluated. Excess acyclovir is present in all the formulations in this example to maximize the permeation driving force.

The permeation of acyclovir from the test formulations through HMS are presented in Table 4 below.

TABLE 4

| | Skin Flux* |
|---|---|
| Non-volatile solvent system | (μg/cm²/h) |
| Polyethylene glycol 400 | 0 |
| Isostearic acid | 0.1 ± 0.09 |
| Isostearic acid + 10% trolamine | 2.7 ± 0.6 |
| Isostearic acid + 30% trolamine | 7 ± 2 |
| Oleic acid | 0.4 ± 0.3 |
| Oleic acid + 10% trolamine | 3.7 ± 0.5 |
| Oleic acid + 30% trolamine | 14 ± 5 |
| Span 80 (sorbitan monooleate) | 0.07 ± 0.03 |
| Ethyl oleate | 0.2 ± 0.2 |
| Ethyl oleate + 10% trolamine | 0.2 ± 0.2 |

*Skin flux measurements represent the mean and standard deviation of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of acyclovir from the above non-volatile solvents are obtained by placing 200 mcL on the stratum corneum side (donor) of hairless mouse skin. The in vitro studies are carried out as described in Example 1. The surprising result showed the polyethylene glycol 400, Span 80, ethyl oleate, or ethyl oleate plus trolamine are not flux-enabling solvents for acyclovir (e.g., steady state flux values significantly less than the steady state flux of acyclovir in the marketed product noted in Table 1, where the flux was about 3 μg /cm²/h). However, the combination of isostearic acid and trolamine or oleic acid and increasing amounts of trolamine are flux-enabling solvents for acyclovir. As can be seen, the highest flux was achieved using 30% trolamine with oleic acid as the non-volatile solvent system.

### Example 3

Formulations of ketoprofen (obtained from Cosma) in various non-volatile solvent systems are evaluated. Excess ketoprofen is present. The permeation of ketoprofen from the test formulations through HMS is presented in Table 5 below.

TABLE 5

| | Skin Flux* |
|---|---|
| Non-volatile solvent system | (μg/cm²/h) |
| Glycerol | 2 ± 1 |
| Polyethylene glycol 400 | 5 ± 2 |
| Span 20 (sorbitan laurate) | 15 ± 3 |
| Propylene glycol | 90 ± 50 |
| Oleic acid | 180 ± 20 |

*Skin flux measurements represent the mean and st. dev of three determinations. Flux measurements reported were determined from the linear region of the cumulative amount versus time plots. The linear region was observed to be between 4-8 hours.. If experimental conditions allowed, the steady-state delivery would likely continue well beyond 8 hours.

Steady state flux of ketoprofen from the above non-volatile solvents are obtained by placing 200 mcL on the stratum corneum side (donor) of hairless mouse skin. The in vitro studies are carried out as described in Example 1. From Table 5, the non-volatile solvents glycerol and polyethylene glycol 400 had low steady state flux values and would not be considered “flux-enabling” (e.g., steady state flux values reported are much lower than the steady state flux value of the marketed product in Table 1, where the flux was about 16 μg/cm²/h). Span 20 would be considered flux-enabling, and propylene glycol or oleic acid provided the highest high flux-enabling non-volatile solvent system.

### Example 4

Formulations of imiquimod (obtained from Yancheng Lvye Chemical Co.) in various non-volatile solvent systems are evaluated. Excess imiquimod is present. The permeation of imiquimod from the test formulations through HMS is presented in Table 6 below.

TABLE 6

| | Skin Flux* |
|---|---|
| Non-volatile solvent system | (μg/cm²/h) |
| Glycerol | 0 |
| Tween 60 (polyoxyethylene | 0.02 ± 0.01 |
| sorbitan monostearate) | |
| Propylene glycol | 0.05 ± 0.02 |
| Span 20 | 0.30 ± 0.05 |
| Isostearic acid | 0.30 ± 0.06 |

*Skin flux measurements represent the mean and st. dev of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of imiquimod from the above non-volatile solvents are obtained by placing 200 mcL on the stratum corneum side (donor) of hairless mouse skin. The in vitro studies are carried out as described in Example 1. From Table 6, the non-volatile solvents glycerol, Tween 60, and propylene glycol had low steady state flux values and would not be considered “flux-enabling” (e.g., steady state flux values reported are much lower than the steady state flux value of the marketed product in Table 1). However, Span 20 and isostearic acid are flux-enabling solvents and are good candidates for evaluation with solid gel-forming forming agents and volatile solvents to design an acceptable solid gel-forming formulation.

### Example 5

Formulations of ropivacaine (obtained from Suzhou Leader Chemical Co.) in various non-volatile solvent systems are evaluated. Excess ropivacaine is present. The permeation of ropivacaine from the test formulations through HMS is presented in Table 7 below.

TABLE 7

| | Skin Flux* |
|---|---|
| Non-volatile solvent system | (μg/cm²/h) |
| Glycerol | 1.2 ± 0.7 |
| Tween 20 (polyoxyethylene | 2.4 ± 0.1 |
| sorbitan monolaurate) | |
| Mineral oil | 8.9 ± 0.6 |
| Isostearic acid | 11 ± 2 |
| Span 20 | 26 ± 8 |

*Skin flux measurements represent the mean and st. dev of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of ropivacaine base from the above non-volatile solvents are obtained by placing 200 mcL on the stratum corneum side (donor) of hairless mouse skin. The in vitro studies are carried out as described in Example 1. From Table 7, the non-volatile solvents glycerol, and Tween 20 had low steady state flux values and would not be considered “flux-enabling” (i.e., steady state flux values reported are much lower than the estimated therapeutic steady state flux value in Table 1, where the flux was about 5 μg/cm²/h). However, mineral oil and isostearic acid are flux-enabling solvents and are good candidates for evaluation with gelling agents and volatile solvents to design an acceptable solid gel-forming formulation. Surprisingly Span 20 has much higher steady state flux values and would qualify as a high flux-enabling solvent.

### Example 6

Formulations of betamethasone dipropionate (BDP) (obtained from Sigma Aldrich) in various non-volatile solvent systems are evaluated. Excess BDP is present. The permeation of BDP from the test formulations through HEM is presented in Table 8 below.

TABLE 8

| | Skin Flux* |
|---|---|
| Non-volatile solvent system | (ng/cm²/h) |
| Propylene glycol | 195.3 ± 68.5 |
| Triacetin | 4.6 ± 2.8 |
| Light mineral oil | 11.2 ± 3.1 |
| Oleic acid | 8.8 ± 3.3 |
| Sorbitan monolaurate | 30.0 ± 15.9 |
| Labrasol | 12.2 ± 6.0 |

*Skin flux measurements represent the mean and st. dev of three determinations. Flux measurements reported were determined from the linear region of the cumulative amount versus time plots. The linear region was observed to be between 6-28 hours. If the experiment was continued it is anticipated the steady state would continue.

Human cadaver skin is used as membrane to select “flux-enabling” solvent for BDP. About 200 mcl of saturated solutions of BDP in various solvents are added to the donor compartment of the Franz cells. In vitro analysis as described in Example 1 is used to determine the steady state flux of BDP. In vitro methodology used is described in Example 1. Active enzymes in the skin convert BMD to betamethasone. The steady state flux values reported in Table 2 are quantified using external betamethasone standards and are reported as amount of betamethasone permeating per unit area and time. As seen from the results, triacetin, labrasol, oleic acid, and light mineral oil have flux values close to the therapeutic sufficient flux of 10 ng/cm²/hr. Addition of gel forming agents and other components could possibly decrease the flux and hence the above mentioned non-volatile solvents may not be an ideal choice as “flux-enabling” solvents. However, sorbitan monolaurate has 3 times higher flux than one possible therapeutic level and hence has better chances to be a “flux-enabling” solvent. Its compatibility with various gelling agents would determine the appropriate levels at which it can be used. Additionally, propylene glycol has 19 times higher flux than therapeutic level needed, and hence provides significantly higher flux than other non-volatile solvent systems tested. The ability of a non-volatile solvent to generate a flux significantly higher than just the minimum “enabling” flux can be advantageous because as the incorporation of other necessary or desired ingredients into the formulation tends to decrease the flux, it may allow achieving the desired therapeutic effect with relatively low drug concentrations in the formulation, which tend to make the formulation less expensive and safer.

### Example 7

Formulations of clobetasol propionate (obtained from Sigma Aldrich) in various non-volatile solvent systems were evaluated. All solvents had 0.1% (w/w) clobetasol propionate. The permeation of clobetasol from the test formulations through HEM is presented in Table 9 below.

TABLE 9

| | Skin Flux* |
|---|---|
| Non-volatile solvent system | (ng/cm²/h) |
| Propylene glycol | 3.8 ± 0.4 |
| Glycerol | 7.0 ± 4.1 |
| Light mineral oil | 31.2 ± 3.4 |
| Isostearic acid (ISA) | 19.4 ± 3.2 |
| Ethyl oleate | 19.4 ± 1.6 |
| Olive oil | 13.6 ± 3.3 |
| Propylene glycol/ISA (9:1) | 764.7 ± 193.9 |

*Skin flux measurements represent the mean and st. dev of three determinations. Flux measurements reported were determined from the linear region of the cumulative amount versus time plots. The linear region was observed to be between 6-28 hours. If the experiment was continued it is anticipated the steady state would continue.

Human cadaver skin is used as a membrane to select “flux-enabling” solvent for clobetasol propionate. In vitro methodology is described in Example 1. About 200 mcl of 0.1% (w/w) solution of clobetasol in various non-volatile solvents is added to the donor compartment of Franz cells. Results obtained after LC analysis are shown in Table 9. All the neat non-volatile solutions studied have an average flux of less than 50 ng/cm²/hr over a 30 hour time period. Propylene glycol and glycerol has the lowest permeation for clobetasol propionate. This result is surprising considering that betamethasone dipropionate which is similar in structure to clobetasol propionate has good flux with propylene glycol. The solvent system which is a mixture of propylene glycol and isostearic acid at a weight ratio of 9:1 has significantly higher flux than either of the solvents alone or the other solvents tested. The average flux is 20 times higher than light mineral oil which appears to be the best non-mixed solvent. Hence, for clobetasol propionate, the propylene glycol/isostearic acid provided the highest flux for a non-volatile solvent system. Among the non-volatile solvents listed in Table 9, only 9:1 propylene glycol:ISA is flux enabling. This is an example of when the flux enabling non-volatile solvent is not a single solvent, but rather a mixture of two or more solvents in designed ratios.

### Example 8

Formulations of diclofenac sodium (obtained from Spectrum) in various non-volatile solvent systems are evaluated. Excess diclofenac sodium is present. The permeation of diclodenac sodium from the test formulations through HMS is presented in Table 10 below.

TABLE 10

| | Skin Flux* |
|---|---|
| Non-volatile solvent system | (μg/cm²/h) |
| Glycerol | 1.7 ± 0.3 |
| Isopropyl myristate | 13 ± 3 |
| Ethyl oleate | 14 ± 4 |
| Propylene glycol | 30 ± 30 |
| Span 20 | 98 ± 20 |

*Skin flux measurements represent the mean and st. dev of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of diclofenac sodium from the above non-volatile solvents are obtained by placing 200 mcL on the stratum corneum side (donor) of hairless mouse skin. The in vitro studies are carried out as described in Example 1. From Table 10, the non-volatile solvent glycerol have steady state flux values comparable to the estimated therapeutic steady state flux value obtained from a marketed product in Table 1 and is considered a flux-enabling solvent. However, the steady state flux values of isopropyl myristate, ethyl oleate, propylene glycol, and Span 20 are at least 10 times the flux value reported for glycerol.

### Example 9

Formulations of diclofenac acid (diclofenac sodium obtained from Spectrum and converted to acid once received) in various non-volatile solvent systems are evaluated. Excess diclofenac acid is present. The permeation of diclofenac from the test formulations through HMS is presented in Table 11 below.

TABLE 11

| | Skin Flux* |
|---|---|
| Non-volatile solvent system | (μg/cm²/h) |
| Glycerol | 0 |
| Isopropyl myristate | 8 ± 3 |
| Ethyl oleate | 7 ± 3 |
| Propylene glycol | 5 ± 2 |
| Span 20 | 3 ± 1 |

*Skin flux measurements represent the mean and st. dev of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of diclofenac acid from the above non-volatile solvents are obtained by placing 200 mcL on the stratum corneum side (donor) of hairless mouse skin. The in vitro studies are carried out as described in Example 1. From Table 11, the non-volatile solvent glycerol has no reported steady state flux value and is not considered a viable non-volatile solvent candidate. However, the steady state flux values of isopropyl myristate, ethyl oleate, propylene glycol, and Span 20 are no more than 10 times the flux value reported for currently available marketed products, and as such, would be considered flux-enabling solvents. It should be noted that the steady state flux values for diclofenac acid from each of the above non-volatile solvents are much lower than the steady state flux values obtained with diclofenac sodium. Therefore, if therapeutically sufficient flux values need to be increased, utilizing a flux-enabling non-volatile solvent and the salt form of diclofenac would likely yield higher steady state flux values than using the acid form of diclofenac.

### Example 10

Formulations of testosterone (obtained from Sigma Aldrich) in various non-volatile solvent systems are evaluated. Excess testosterone is present. The permeation of testosterone from the test formulations through HMS is presented in Table 12 below.

TABLE 12

| | Skin Flux* |
|---|---|
| Non-volatile solvent system | (μg/cm²/h) |
| Tween 60 | 0 |
| Span 20 | 1.4 ± 0.2 |
| Polyethylene glycol 400 | 1.2 ± 0.1 |
| Isostearic acid | 2.6 ± 0.1 |
| Propylene glycol | 6 ± 2 |

*Skin flux measurements represent the mean and st. dev of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of testosterone from the above non-volatile solvents are obtained by placing 200 mcL on the stratum corneum side (donor) of hairless mouse skin. The in vitro studies are carried out as described in Example 1. From Table 12, the non-volatile solvent Tween 60 has no reported steady state flux value and s not considered a viable non-volatile solvent candidate. However, the steady state flux values of Span 20, polyethylene glycol 400, isostearic acid, and propylene glycol have steady state flux values comparable to currently available marketed products, and thus, would be considered flux-enabling solvents. However, although all the non-volatile solvents except for Tween 60 are flux-enabling, propylene glycol may be better for a practical formulation because the high flux generated by it means the same amount of drug can be delivered with smaller skin contact area.

### Example 11

Formulations of hydromorphone HCl (obtained from Johnson Matthey) in various non-volatile solvent systems are evaluated. Excess hydromorphone HCl is present. The permeation of hydromorphone HCl from the test formulations through HMS is presented in Table 13 below.

TABLE 13

| | Skin Flux* |
|---|---|
| Non-volatile solvent system | (μg/cm²/h) |
| Propylene glycol | 2 ± 0.8 |
| Isostearic acid | 3 ± 3 |
| Ethyl oleate | 40 ± 16 |

*Skin flux measurements represent the mean and st. dev of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of hydromorphone from the above non-volatile solvents are obtained by placing 200 mcL on the stratum corneum side (donor) of hairless mouse skin. The in vitro studies are carried out as described in Example 1. From Table 13, the non-volatile solvents propylene glycol and isostearic acid may qualify as flux-enabling solvents (based on an estimated therapeutically sufficient flux for hydromorphone is 2 μg/cm2/h). Clearly, the steady state flux value of hydromorphone from ethyl oleate is much higher and would qualify as a high flux-enabling solvent.

### Example 12

Formulations of hydromorphone (salt form obtained from Johnson Matthey and converted to base form once received) in various non-volatile solvent systems are evaluated. Excess hydromorphone is present. The permeation of hydromorphone from the test formulations through HMS is presented in Table 14 below.

TABLE 14

| | Skin Flux* |
|---|---|
| Non-volatile solvent system | (μg/cm²/h) |
| Propylene glycol | 1 ± 1 |
| Isostearic acid | 7 ± 2 |
| Ethyl oleate | 6 ± 2 |

*Skin flux measurements represent the mean and st dev of three determinations. Flux measurements reported were determined from the linear region of the cumulative amount versus time plots. The linear region was observed to be between 4-8 hours. If experimental conditions allowed, the steady-state delivery would likely continue well beyond 8 hours.

Steady state flux of hydromorphone from the above non-volatile solvents are obtained by placing 200 μL on the stratum corneum side (donor) of hairless mouse skin. The in vitro studies are carried out as described in Example 1. From Table 14, the non-volatile solvent propylene glycol may qualify as flux-enabling solvents (based on an estimated therapeutically sufficient flux for hydromorphone is 2 μg/cm2/h). The steady state flux value of hydromorphone from isostearic acid and ethyl oleate would also qualify as flux-enabling solvents.

### Examples 13-17

Prototype solid gel-forming formulations are prepared as follows. Several solid gel-forming formulations are prepared in accordance with embodiments of the present invention in accordance with Table 15, as follows:

TABLE 15

| Example | | | | |
|---|---|---|---|---|
| 13 | 14 | 15 | 16 | 17 |

% by weight

Volatile Solvents

| | | | | | |
|---|---|---|---|---|---|
| Ethanol | 25 | 21 | 24 | 18.5 | 43 |
| Water | | 32 | | 28 | 22 |

Gelling Agents

| | | | | | |
|---|---|---|---|---|---|
| Eudragit RL-PO | 18 | | 40 | | |
| Eudragit E-100 | | | | 18.5 | |
| Polyvinyl alcohol | | 21 | | 18.5 | 14 |

Non-volatile solvents

| | | | | | |
|---|---|---|---|---|---|
| Glycerol | | | 12 | | 14 |
| Propylene glycol | | 21 | 4 | | |
| Polyethylene glycol | | | | | 6 |
| Isostearic acid | 36 | | 13 | | |
| Span 20 | | | | 11 | |
| Trolamine | 18 | | 4 | | |

Drug

| | | | | | |
|---|---|---|---|---|---|
| Acyclovir | 3 | | | | |
| Ketoprofen | | 5 | | | |
| Imiquimod | | | | | |
| Ropivacaine | | | 3 | | |
| Diclofenac Na | | | | 5.5 | |
| Testosterone | | | | | 1 |

Gel formulation of Examples 13-17 are prepared in the following manner:
The gelling agents are dissolved in the volatile solvent (e.g., dissolve polyvinyl alcohol in water, Eudragit polymers in ethanol),
The non-volatile solvent(s) is mixed with the gelling agent/volatile solvent mixture.
The resulting solution is vigorously mixed for several minutes.
The drug is then added and the formulation is mixed again for several minutes.

In all the Examples noted above, the flux-enabling non-volatile solvent/gelling agent/volatile solvent combination is compatible as evidenced by a homogeneous, single phase system that exhibited appropriate drying time, and provided a stretchable solid gel layer and steady state flux for the drug (see Example 18 below).

### Example 18

The formulations of the examples are tested in a hairless mouse skin (HMS) or HEM in vitro model described in Example 1. Table 16 shows data obtained using the experimental process outlined above.

TABLE 16
Steady-state flux (J)

| | J* |
|---|---|
| Formulation | (μg/cm²/h) |
| Example 13 | 19 ± 1*** |
| Example 14 | 35 ± 20*** |
| Example 15 | 32 ± 2*** |
| Example 16** | 5 ± 2**** |
| Example 17 | 4 ± 1*** |

*Skin flux measurements represent the mean and st. dev of three determinations.
**Data gathered using human epidermal membrane.
***Flux measurements reported were determined from the linear region of the cumulative amount versus time plots. The linear region was observed to be between 4-8 hours. If experimental conditions allowed, the steady-state delivery would likely continue well beyond 8 hours.
****Flux measurements reported were determined from the linear region of the cumulative amount versus time plots. The linear region was observed to be between 6-28 hours. If the experiment was continued it is anticipated the steady state would continue.

Acyclovir, ropivacaine, and testosterone have surprisingly higher steady state flux values when the flux-enabling non-volatile solvent is incorporated into the solid gel-forming formulation. It is speculated that the higher flux values may be the result of contributions of the volatile solvent or the gelling agent impacting the chemical environment (e.g., increasing solubility) of the drug in the formulation resulting in higher flux values. Conversely, ketoprofen and diclofenac have lower steady state flux values when the enabling non-volatile solvent is incorporated into the formulation. This could be the result of the volatile solvent system or gelling agent having the opposite impact on the chemical environment (e.g., decreasing solubility, physical interactions between drug and other ingredients of the formulation) resulting in lower flux values. The steady state flux value for imiquimod is unchanged when comparing the solid gel-forming formulation with the flux-enabling non-volatile solvent flux values.

### Example 19

A formulation with the following composition: 10.4% polyvinyl alcohol, 10.4% polyethylene glycol 400, 10.4% polyvinyl pyrrolidone K-90, 10.4% glycerol, 27.1% water, and 31.3% ethanol was applied onto a human skin surface at an elbow joint and a finger joint, resulting in a thin, transparent, flexible, and stretchable film. After a few minutes of evaporation of the volatile solvents (ethanol and water), a solidified gel layer that was peelable and washable was formed. The stretchable film had good adhesion to the skin and did not separate from the skin on joints when bent, and could easily be peeled away from the skin.

### Examples 20-22

Three formulations similar to the formulation in Example 15 (replacing ropivacaine base with ropivacaine HCl) are applied on the stratum corneum side of freshly separated hairless mouse skin. The in vitro flux is determined for each formulation as outlined in Example 1. The formulation compositions are noted in Table 17 below.

TABLE 17

| Example | | |
|---|---|---|
| 20 | 21 | 22 |

% by weight

| | | | |
|---|---|---|---|
| PVA | 15 | 15 | 15 |
| Water | 23 | 23 | 23 |
| Ethylcellulose N-100 | 11 | 11 | 11 |
| Ethanol | 33 | 33 | 33 |
| Span 20 | 11 | | |
| Polyethylene glycol 400 | | 11 | |
| Tween 40 | | | 11 |
| Tromethamine | 4 | 4 | 4 |
| Ropivacaine HCl | 3 | 3 | 3 |
| Avg. Flux* (μg/cm2/h) | 15 ± 1 | 4.7 ± 0.3 | 3.4 ± 0.7 |

*Flux values represent the mean and st dev of three determinations. Flux measurements reported were determined from the linear region of the cumulative amount versus time plots. The linear region was observed to be between 6-31 hours. If the experiment was continued it is anticipated the steady state would continue.

All three formulations have the exact same compositions of gelling agent, volatile solvents, and flux-enabling non-volatile solvent. Since the only difference is which flux-enabling non-volatile solvent is used, it is reasonable to conclude that for ropivacaine HCl that Span 20, polyethylene glycol 400, and Tween 40 each qualify as flux-enabling non-volatile solvents.

### Examples 23-28

### Adhesive Gel Forming Formulations with Clobetasol Propionate

Adhesive solid gel forming formulations containing 0.05% (w/w) clobetasol propionate with propylene glycol and isostearic acid as non-volatile solutions and various gel formers are prepared from the ingredients shown in Table 18.

TABLE 18

| | | | % | % | |
|---|---|---|---|---|---|
| Example/ | % | % | Propylene | Isostearic | % |
| Polymer | Polymer | Ethanol | glycol | acid | Water |
| 23/Polyvinyl alcohol | 20 | 30 | 19.6 | 0.4 | 30 |
| 24/Shellac | 50 | 30 | 19.6 | 0.4 | 0 |
| 25/Dermacryl 79 | 65.80 | 21.18 | 12.76 | 0.26 | 0 |
| 26/Eudragit E100 | 50 | 30 | 19.6 | 0.40 | 0 |
| 27/Eudragit RLPO | 50 | 30 | 19.6 | 0.40 | 0 |
| 28/Gantrez S97 | 14.3 | 57.1 | 28 | 0.6 | 0 |

Each of the compositions shown above is studied for flux of clobetasol propionate as shown in Table 19 as follows:

TABLE 19
Steady state flux of Clobetasol propionate through human
cadaver skin at 35° C.

| | J* |
|---|---|
| Formulation | (ng/cm²/h) |
| Example 23 | 87.8 ± 21.4 |
| Example 24 | 9.7 ± 2.4 |
| Example 25 | 8.9 ± 0.8 |
| Example 26 | 3.2 ± 1.7 |
| Example 27 | 20.2 ± 18.6 |
| Example 28 | 147.5 ± 38.8 |

*Skin flux measurements represent the mean and st. dev of three determinations. Flux measurements reported are determined from the linear region of the cumulative amount versus time plots. The linear region are observed to be between 6-28 hours. If the experiment is continued, it is anticipated the steady state would continue.

As seen from Table 19 formulation described in Example 23 that contained polyvinyl alcohol as gelling agent has high flux of clobetasol propionate. Polyvinyl alcohol is known to form stretchable films and it is likely that this formulation will have acceptable wear properties. The toughness of the resulting solid gel can be modified by adding appropriate plasticizers if needed. Tackiness can also be modified by adding appropriate level of a tackifier or by adding appropriate level of another gel forming agent such as dermacryl 79.

Regarding formulation described in Example 28, higher levels of ethanol are needed to dissolve the polymer. The formulation has the highest flux of clobetasol propionate among the gelling agents studied. The wear properties of this formulation can be modified by adding appropriate levels of other ingredients including but not limited to plasticizers, tackifiers, non-volatile solvents and or gelling agents. The formulation can be removed by washing it with ethanol, or another appropriate solvent, and washing with a medium amount of force.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. An adhesive solid gel-forming formulation for dermal delivery of a drug, comprising:
a) a drug;
b) a solvent vehicle, comprising:
i) a volatile solvent system including one or more volatile solvent, and
ii) a non-volatile solvent system including one or more non-volatile solvents, wherein at least one non-volatile solvent is a flux-enabling non-volatile solvent for said drug; and
wherein the formulation has a viscosity suitable for application and adhesion to a skin surface prior to evaporation of the volatile solvent system, and wherein the formulation applied to the skin surface forms a solidified gel layer after at least partial evaporation of the volatile solvent system, wherein the drug continues to be dermally delivered after the volatile solvent system is substantially evaporated.

2. A formulation as in claim 1, further comprising a gelling agent.

3. A formulation as in claim 1, further comprising a permeation enhancing agent.

4. A formulation as in claim 1, wherein the non-volatile solvent system acts as a plasticizer for said gelling agent.

5. A formulation as in claim 1, wherein said volatile solvent system comprises water.

6. A formulation as in claim 1, wherein said volatile solvent system comprises water and ethanol.

7. A formulation as in claim 1, wherein said volatile solvent system comprises water and propyl alcohol.

8. A formulation as in claim 1, wherein said volatile solvent system comprises at least one solvent more volatile than water, and is selected from the group consisting of ethyl ether, iso-amyl acetate, denatured alcohol, methanol, ethanol, isopropyl alcohol, propanol, C4-C6 hydrocarbons including butane isobutene, pentane, and hexane, acetone, chlorobutanol, ethyl acetate, fluro-chloro-hydrocarbons, turpentine, cytopentasiloxane, cyclomethicone, methyl ethyl ketone, mixtures thereof, and mixtures with water thereof.

9. A formulation as in claim 1, wherein the flux-enabling non-volatile solvent is a flux-enabling, plasticizing non-volatile solvent.

10. A formulation as in claim 1, wherein the flux-enabling non-volatile solvent provides at least twice the flux for a particular drug when present in the non-volatile solvent system alone than is necessary to achieve a therapeutically sufficient flux.

11. A formulation as in claim 1, wherein the non-volatile solvent system comprises one or more solvents selected from the group consisting of 1,2,6-hexanetriol, alkyltriols, alkyldiols, acetyl monoglycerides, tocopherols, alkyl dioxolanes, p-propenylanisole, dimethyl isosorbide, alkyl glucoside, benzoic acid, benzyl alcohol, butyl alcohol, beeswax, benzyl benzoate, butylene glycol, caprylic/capric triglyceride, caramel, cinnamaldehyde, cocoa butter, cocoglycerides, corn syrup, cresol, cyclomethicone, diacetin, diacetylated monoglycerides, dibutyl sebecate, diethanolamine, diethylene glycol monoethyl ether, diglycerides, dipropylene glycol, ethylene glycol, eugenol, fat, fatty acid (esters glycerides), fatty alcohols, liquid sugars, ginger extract, glycerin, high fructose corn syrup, IPM, IP palmitate, isostearic acidlimonene, milk, mineral oil, monoacetin, monoglycerides, oleic acid, octyldodecanol, oleyl alcohol, PEG (propylene glycols), vegetable oils including, olive alcohol, palm oil, corn oil, cottonseed oil, cinnamon oil, clove oil, coconut oil, anise oil, apricot oil, coriander oil, cassia oil, castor oil, lemon oil, lime oil, pine needle oil, sesame oil, spearmint oil, soybean oil, eucalyptus oil, hydrogenated castor oil, orange oil, nutmeg oil, peanut oil, peppermint oil, petrolatum, phenol, polypropylene glycol, propylene glycol, trolamine, tromethemine, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters including PEG-stearates, PEG-oleates, PEG-laurates, PEG fatty acid diesters including PEG-dioleates, PEG-distearates, PEG-castor oils, glyceryl behenate, PEG glycerol fatty acid esters including PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid multisterol extract, myristyl alcohol, neutral oil, PEG-octyl phenyl ethers, PEG-alkyl ethers including PEG-cetyl ethers, PEG-stearyl ethers, PEG-sorbitan fatty acid esters including PEG-sorbitan diisosterates, PEG-sorbitan monostearates, propylene glycol fatty acid esters including propylene glycol stearates, propylene glycol caprylate/caprates, sodium pyrrolidone carboxylate, sorbitol, squalene, stear-o-wet, triacetin, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty alcohol ethers, alkyl-amides (N,N-dimethylalkylamides), N-methyl pyrrolidone related compounds, sorbitan fatty acid surfactants including sorbitan monooleate, sorbitan trioleate, sorbitan monopalmitate, ethyl oleate, polyglycerized fatty acids, glycerol monooleate, glyceryl monomyristate, glycerol esters of fatty acids, and mixtures thereof.

12. A formulation as in claim 2, wherein the gelling agent is selected from the group consisting of: ammonia methacrylate, carrageenan, cellulose acetate phthalate aqueous, carboxy methyl cellulose Na, carboxy polymethylene, cellulose, cellulose acetate (microcrystalline), cellulose polymers, divinyl benzene styrene, ethyl cellulose, ethylene vinyl acetate, silicone, polyisobutylene, Shellac (FMC BioPolymer), guar gum, guar rosin, cellulose derivatives including hydroxy ethyl cellulose hydroxy methyl cellulose, hydroxy propyl cellulose, hydroxypropyl methyl cellulose, carboxymethyl cellulose, methyl cellulose, hypromellose phthalate, methyl acrylate, microcrystalline wax, polyvinyl alcohol, polyvinyl acetate, polyvinyl acetate phthalate, ethyl cellulose, polyvinyl pyrrolidone (PVP), acrylate, PEG/PVP, xanthan gum, trimethyl siloxysilicate, maleic acid/anhydride copolymersl, polacrilin, poloxamer, polyethylene oxide, poly glactic acid/poly-l-lactic acid, turpene resin, locust bean gum, prolamine (Zein), acrylic copolymers, polyurethane dispersions, gelatin, dextrin, starch, polyvinyl alcohol-polyethylene glycol copolymers, methyacrylic acid-ethyl acrylate copolymers, methacrylic acid and methacrylate based polymers including poly(methacrylic acid) copolymers and methylmethacrylate copolymers, esters of polyvinylmethylether/maleic anhydride copolymers, and combinations thereof.

13. A formulation as in claim 2, wherein the gelling agent includes a member selected from the group consisting of shellac, polyvinyl acetate phthalate, polyvinyl alcohol, polyvinyl pyrrolidone, carrageenin, gelatin, dextrin, gelatin, guar gum, polyethylene oxide having a weight average molecular weight greater than about 5,000 Mw, starch, xantham gum, cellulose derivatives, polyvinyl alcohol-polyethylene glycol copolymers and methyacrylic acid-ethyl acrylate copolymers, methacrylic acid and methacrylate based polymers including poly(methacrylic acid) copolymers and methylmethacrylate copolymers, aminoalkyl methacrylate copolymers ammonioalkyl methacrylate copolymers, butyl methacrylate-methyl methacrylate copolymers, acrylates/octylacrylamide copolymers, and mixtures thereof.

14. A formulation as in claim 2, wherein the gelling agent includes a cellulose derivative selected from the group consisting of hydroxyethylcellulose, ethylcellulose, carboxymethylcellulose, hydroxypropylcellulose, copolymers of methyl vinyl ether and maleic anhydride, and mixtures thereof.

15. A formulation as in claim 2, wherein the gelling agent is selected from the group consisting of polyvinyl alcohol-polyethylene glycol copolymers, methacrylic acid and methacrylate-based copolymers including poly(methacrylic acid) copolymers, methylmethacrylate copolymers, methyacrylic acid-ethyl acrylate copolymers, and mixtures thereof.

16. A formulation as in claim 1, wherein the drug is selected from the group consisting of non-steroidal anti-inflammatory drugs (NSAIDs) including ketoprofen and diclofanec; COX-2 selective NSAIDs and agents; COX-3 selective NSAIDs and agents; local anesthetics including lidocaine, bupivacaine, ropivacaine, and tetracaine; steroids including clobetasol propionate, halobetasol propionate, betamethasone dipropionate, dexamethasone; antibiotics, retinoids, clonidine, peroxides, retinol, salicylic acid, imiquimod, humectants, emollients, antiviral drugs including acyclovir, penciclovir, famciclovir, valacyclovir, steroids, and behenyl alcohol; and combinations thereof.

17. A formulation as in claim 1, wherein the drug is a humectant or emollient.

18. A formulation as in claim 1, wherein the drug is suitable for treating a herpes infection, muscle skeletal pain, diaper rash, fungal infection, nicotine addition or smoking cessation, histamine response (anti-histamine), viral infection (anti-viral), dermatitis, infection, psoriasis, eczema, acne, sex steroid deficiency, neuropathic pain, warts, and combinations thereof.

19. A formulation as in claim 1, wherein the drug is selected from the group consisting of a corticosteroid, sex steroid, anti-histamine, anti-viral, nicotine, an immune modulating agent, vitamin D or a vitamin D derivative, retinoic acid or a derivative of retinoic acid, local anesthetic, and combinations thereof.

20. A formulation as in claim 1, wherein the solidified gel layer is sufficiently flexible and adhesive to the skin such that when applied to the skin at a human joint or to a curved body surface, the solidified gel layer will remain substantially intact on the skin upon bending of the joint or the bending or stretching of the curved body surface.

21. A formulation as in claim 1, wherein the formulation is configured to deliver the drug at a therapeutically effective rate for at least about 2 hours following the formation of said solidified gel layer.

22. A formulation as in claim 1, wherein the formulation is configured to deliver the drug at a therapeutically effective rate for at least about 12 hours following the formation of said solidified gel layer.

23. A formulation as in claim 2, wherein the gelling agent is dispersed or solvated in the solvent vehicle.

24. A formulation as in claim 1, wherein the weight ratio of the non-volatile solvent system to the gelling agent is from about 0.01:1 to about 10:1.

25. A formulation as in claim 1, wherein the volatile solvent system is capable of causing human skin irritation and at least one non-volatile solvent of said non-volatile solvent system is capable of reducing the skin irritation.

26. A formulation as in claim 1, wherein the solidified gel layer is formed within about 15 minutes of application to the skin surface under standard skin and ambient conditions.

27. A formulation as in claim 1, wherein the formulation has an initial viscosity prior to skin application from about 100 to about 3,000,000 centipoises.

28. A formulation as in claim 1, wherein the weight percentage of the volatile solvent system is from about 2 wt % to about 50 wt %.

29. A formulation as in claim 1, wherein the non-volatile solvent system includes multiple non-volatile solvents, and at least one of the non-volatile solvents is capable of improving the compatibility of the non-volatile solvent system with the gelling agent.

30. A formulation as in claim 1, wherein the non-volatile solvent includes at least two non-volatile solvents, and wherein one of said at least two non-volatile solvents is included to improve compatibility with the gelling agent.

31. A formulation as in claim 1, wherein the solidified formulation can be removed by washing with either water or other preferred washing solvents.

32. A method of dermally delivering a drug, comprising:
a) applying an adhesive solid gel-forming formulation to a skin surface of a subject, said adhesive solid gel-forming formulation, comprising:
i) a drug;
ii) a solvent vehicle, comprising:
a volatile solvent system including one or more volatile solvents, and
a non-volatile solvent system including one or more non-volatile solvent, wherein at least one non-volatile solvent is a flux-enabling non-volatile solvent for said drug;
wherein the formulation has a viscosity suitable for application and adhesion to a skin surface prior to evaporation of the volatile solvent system, and wherein the formulation applied to the skin surface forms a solidified gel layer after at least partial evaporation of the volatile solvent system, wherein the drug continues to be dermally delivered after the volatile solvent system is substantially evaporated; and
b) dermally delivering the drug from the solidified gel layer to the subject at therapeutically effective rates over a sustained period of time.

33. A method as in claim 32, wherein the step of applying includes applying the adhesive solid gel-forming formulation at a thickness from about 0.01 mm to about 2 mm.

34. The method as in claim 32, wherein the formulation further comprises a gelling agent.

35. A method as in claim 32, wherein the non-volatile solvent system acts as a plasticizer for said gelling agent.

36. A method as in claim 32, wherein said volatile solvent system comprises water.

37. A method as in claim 32, wherein said volatile solvent system comprises at least one solvent more volatile than water, and is selected from the group consisting of ethyl ether, iso-amyl acetate, denatured alcohol, methanol, ethanol, isopropyl alcohol, propanol, C4-C6 hydrocarbons, butane isobutene, pentane, hexane, acetone, chlorobutanol, ethyl acetate, fluro-chloro-hydrocarbons, turpentine, cytopentasiloxane, cyclomethicone, methyl ethyl ketone, mixtures thereof, and mixtures with water thereof.

38. A method as in claim 32, wherein the flux-enabling non-volatile solvent is a flux-enabling, plasticizing non-volatile solvent.

39. A method as in claim 32, wherein the flux-enabling non-volatile solvent is provides at least twice the flux for a particular drug when present in the non-volatile solvent system alone than is necessary to achieve a therapeutically sufficient flux.

40. A method as in claim 32, wherein the non-volatile solvent system comprises one or more solvents selected from the group consisting of 1,2,6-hexanetriol, alkyltriols, alkyldiols, acetyl monoglycerides, tocopherols, alkyl dioxolanes, p-propenylanisole, dimethyl isosorbide, alkyl glucoside, benzoic acid, benzyl alcohol, butyl alcohol, beeswax, benzyl benzoate, butylene glycol, caprylic/capric triglyceride, caramel, cinnamaldehyde, cocoa butter, cocoglycerides, corn syrup, cresol, cyclomethicone, diacetin, diacetylated monoglycerides, dibutyl sebecate, diethanolamine, diethylene glycol monoethyl ether, diglycerides, dipropylene glycol, ethylene glycol, eugenol, fat, fatty acid (esters glycerides), fatty alcohols, liquid sugars, ginger extract, glycerin, high fructose corn syrup, IPM, IP palmitate, isostearic acidlimonene, milk, mineral oil, monoacetin, monoglycerides, oleic acid, octyldodecanol, oleyl alcohol, PEG (propylene glycols), vegetable oils including, olive alcohol, palm oil, corn oil, cottonseed oil, cinnamon oil, clove oil, coconut oil, anise oil, apricot oil, coriander oil, cassia oil, castor oil, lemon oil, lime oil, pine needle oil, sesame oil, spearmint oil, soybean oil, eucalyptus oil, hydrogenated castor oil, orange oil, nutmeg oil, peanut oil, peppermint oil, petrolatum, phenol, polypropylene glycol, propylene glycol, trolamine, tromethemine, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters including PEG-stearates, PEG-oleates, PEG-laurates, PEG fatty acid diesters including PEG-dioleates, PEG-distearates, PEG-castor oils, glyceryl behenate, PEG glycerol fatty acid esters including PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid multisterol extract, myristyl alcohol, neutral oil, PEG-octyl phenyl ethers, PEG-alkyl ethers including PEG-cetyl ethers, PEG-stearyl ethers, PEG-sorbitan fatty acid esters including PEG-sorbitan diisosterates, PEG-sorbitan monostearates, propylene glycol fatty acid esters including propylene glycol stearates, propylene glycol caprylate/caprates, sodium pyrrolidone carboxylate, sorbitol, squalene, stear-o-wet, triacetin, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty alcohol ethers, alkyl-amides (N,N-dimethylalkylamides), N-methyl pyrrolidone related compounds, sorbitan fatty acid surfactants including sorbitan monooleate, sorbitan trioleate, sorbitan monopalmitate, ethyl oleate, polyglycerized fatty acids, glycerol monooleate, glyceryl monomyristate, glycerol esters of fatty acids, and mixtures thereof.

41. A method as in claim 35, wherein the gelling agent is selected from the group consisting of ammonia methacrylate, carrageenan, cellulose acetate phthalate aqueous, carboxy methyl cellulose Na, carboxy polymethylene, cellulose, cellulose acetate (microcrystalline), cellulose polymers, divinyl benzene styrene, ethyl cellulose, ethylene vinyl acetate, silicone, polyisobutylene, Shellac (FMC BioPolymer), guar gum, guar rosin, cellulose derivatives including hydroxy ethyl cellulose hydroxy methyl cellulose, hydroxy propyl cellulose, hydroxypropyl methyl cellulose, carboxymethyl cellulose, methyl cellulose, hypromellose phthalate, methyl acrylate, microcrystalline wax, polyvinyl alcohol, polyvinyl acetate, polyvinyl acetate phthalate, PVP ethyl cellulose, polyvinyl pyrrolidone (PVP), acrylate, PEG/PVP, xanthan gum, trimethyl siloxysilicate, maleic acid/anhydride copolymersl, polacrilin, poloxamer, polyethylene oxide, poly glactic acid/poly-l-lactic acid, turpene resin, locust bean gum, prolamine (Zein), acrylic copolymers, polyurethane dispersions, gelatin, dextrin, starch, polyvinyl alcohol-polyethylene glycol copolymers, methyacrylic acid-ethyl acrylate copolymers, methacrylic acid and methacrylate based polymers including poly(methacrylic acid) copolymers and methylmethacrylate copolymers, including Rohm and Haas' Eudragit polymers (Eudragit (E, L, NE, RL, RS, S100)), esters of polyvinylmethylether/maleic anhydride copolymer, and combinations thereof.

42. A method as in claim 35, wherein the gelling agent includes a member selected from the group consisting of shellac, poly vinyl acetate phthalate, polyvinyl alcohol, polyvinyl pyrrolidone, carrageenin, gelatin, dextrin, gelatin, guar gum, polyethylene oxide having a weight average molecular weight greater than about 5,000 Mw, starch, xantham gum, cellulose derivatives, polyvinyl alcohol-polyethylene glycol copolymers and methyacrylic acid-ethyl acrylate copolymers, methacrylic acid and methacrylate based polymers including poly(methacrylic acid) copolymers and methylmethacrylate copolymers, aminoalkyl methacrylate copolymers ammonioalkyl methacrylate copolymers, butyl methacrylate-methyl methacrylate copolymers, acrylates/octylacrylamide copolymers, and mixtures thereof.

43. A method as in claim 35, wherein the gelling agent includes a cellulose derivative selected from the group consisting of hydroxyethylcellulose, ehtylcellulose, carboxymethylcellulose, hydroxypropylcellulose, copolymers of methyl vinyl ether and maleic anhydride, and mixtures thereof.

44. A method as in claim 35, wherein the gelling agent is selected from the group consisting of polyvinyl alcohol-polyethylene glycol copolymers, methacrylic acid and methacrylate-based copolymers including poly(methacrylic acid) copolymers, methylmethacrylate copolymers, methyacrylic acid-ethyl acrylate copolymers, and mixtures thereof.

45. A method as in claim 32, wherein the drug is selected from the group consisting of non-steroidal anti-inflammatory drugs (NSAIDs) including ketoprofen and diclofanec; COX-2 selective NSAIDs and agents; COX-3 selective NSAIDs and agents; local anesthetics including lidocaine, bupivacaine, ropivacaine, and tetracaine; steroids including clobetasol propionate, halobetasol propionate, betamethasone dipropionate, dexamethasone; antibiotics, retinoids, clonidine, peroxides, retinol, salicylic acid, imiquimod, humectants, emollients, antiviral drugs including acyclovir, penciclovir, famciclovir, valacyclovir, steroids, and behenyl alcohol; and combinations thereof.

46. A method as in claim 32, wherein the drug is a humectant or emollient.

47. A method as in claim 32, wherein the drug is suitable for treating a herpes infection, muscle skeletal pain, diaper rash, fungal infection, nicotine addition or smoking cessation, histamine response (anti-histamine), viral infection (anti-viral), dermatitis, infection, psoriasis, eczema, acne, sex steroid deficiency, neuropathic pain, warts, and combinations thereof.

48. A method as in claim 32, wherein the drug is selected from the group consisting of a corticosteroid, sex steroid, anti-histamine, anti-viral, nicotine, an immune modulating agent, vitamin D or a vitamin D derivative, retinoic acid or a derivative of retinoic acid, local anesthetic, and combinations thereof.

49. A method as in claim 32, wherein the solidified gel layer is sufficiently flexible and adhesive to the skin such that when applied to the skin at a human joint or to a curved body surface, the solidified gel layer will remain substantially intact on the skin upon bending of the joint or the bending or stretching of the curved body surface.

50. A method as in claim 32, wherein the formulation is configured to deliver the drug at a therapeutically effective rate for at least about 2 hours following the formation of said solidified gel layer.

51. A method as in claim 32, wherein the formulation is configured to deliver the drug at a therapeutically effective rate for at least about 12 hours following the formation of said solidified gel layer.

52. A method as in claim 32, wherein the gelling agent is dispersed or solvated in the solvent vehicle.

53. A method as in claim 32, wherein the weight ratio of the non-volatile solvent system to the gelling agent is from about 0.01:1 to about 10:1.

54. A method as in claim 32, wherein the volatile solvent system is capable of causing human skin irritation and at least one non-volatile solvent of said non-volatile solvent system is capable of reducing the skin irritation.

55. A method as in claim 32, wherein the solidified gel layer is formed within about 15 minutes of application to the skin surface under standard skin and ambient conditions.

56. A formulation as in claim 1, wherein the formulation has an initial viscosity prior to skin application from about 100 to about 3,000,000 centipoises.

57. A method as in claim 32, wherein the weight percentage of the volatile solvent system is from about 2 wt % to about 50 wt %.

58. A method as in claim 32, wherein the non-volatile solvent system includes multiple non-volatile solvents, and at least one of the non-volatile solvents is capable of improving the compatibility of the non-volatile solvent system with the gelling agent.

59. A method as in claim 32, wherein the non-volatile solvent includes at least two non-volatile solvents, and wherein one of said at least two non-volatile solvents is included to improve compatibility with the gelling agent.

60. A method as in claim 32, wherein the solidified formulation can be removed by washing with either water or other preferred washing solvents.

61. A solidified gel layer for delivering a drug, comprising:
a) a drug;
b) a non-volatile solvent system including one or more non-volatile solvent, wherein at least one non-volatile solvent is a flux-enabling non-volatile solvent for said drug;
wherein said solidified gel layer can be stretched in at least one direction by 5% without breaking or cracking.
